(19) United States
(12) Patent Application Publication
(10) Pub. No.: US 2008/0206871 A1
(43) Pub. Date: Aug. 28, 2008

(54) Large-Scale Production of Recombinant Transmembrane and Cytosolic Proteins

(76) Inventors: Julio L. Vergara, Los Angeles, CA (US); Marino G. Di Franco, Santa Monica, CA (US)

Correspondence Address: DLA PIPER US LLP, 4365 EXECUTIVE DRIVE, SUITE 1100, SAN DIEGO, CA, 92121-2133, US

(21) Appl. No.: 11/664,832

(22) PCT Filed: Oct. 7, 2005
(86) PCT No.: PCT/US2005/036225

§ 371 Date: Nov. 8, 2007

Related U.S. Application Data
(60) Provisional application No. 60/617,516, filed on Oct. 8, 2004.

(51) Int. Cl. C12N 15/87; C12N 5/10

(52) U.S. Cl. 435/461; 435/366

(57) ABSTRACT

The present invention provides a method for producing large quantities of transmembrane and cytosolic proteins in a mammalian muscle cell. The method involves transfecting skeletal muscle cells in vivo with nucleic acids encoding the proteins by electroporation. The invention results in production of the desired protein on the order of about 2-3 magnitudes more as compared to standard methods, allowing for various biological uses including purification and crystallization.

### CROSS REFERENCE TO RELATED APPLICATION

This application claims the benefit of priority under 35 U.S.C. § 120 of U.S. Application Ser. No. 60/617,516, filed Oct. 8, 2004, which is incorporated by reference in its entirety in the disclosure of this application.

### GRANT INFORMATION

This invention was made with government support under Grant Nos.: AR47664, AR25201 and GM074706 awarded by the National Institutes of Health. The United States government has certain rights in this invention.

### BACKGROUND OF THE INVENTION

1. Field of the Invention

The present invention relates generally to large-scale production of proteins, and more specifically, to methods for producing heterologous transmembrane and cytosolic proteins in mammalian skeletal muscle cells.

2. Background Information

The production of mammalian proteins in sufficient quantity and quality for structural and functional studies is a major challenge in biology. Prokaryotic expression systems, though powerful in their ability to generate massive quantities of recombinant proteins, have severe limitations for the expression of properly folded (and processed) full-length replicas of a large number of eukaryotic proteins, including both transmembrane and cytosolic (soluble) proteins; in particular, ion channels and transporters. An alternative expression system which approximates the yield of bacteria, but at the same time overcome some of their limitations, is necessary.

### SUMMARY OF THE INVENTION

The present invention is based on the discovery that skeletal muscles have unique characteristics that make them ideal to serve in a method for producing eukaryotic transmembrane and cytosolic proteins of interest. The invention methods involve transfecting skeletal muscle cells in vivo with DNA encoding such proteins of interest. This process results in production of the desired protein of interest by the transfected muscles in sufficient large quantities for use in other protein studies.

In one embodiment, there is provided an in vivo method for producing about 2-3 orders of magnitude more transmembrane protein in a mammalian cell as compared to standard methods by contacting a nucleic acid sequence encoding the transmembrane protein and operably linked to regulatory elements with a skeletal muscle cell of a subject, and introducing the nucleic acid sequence into the cell using electroporation, wherein expression of the transmembrane protein is by endogenous translation of the nucleic acid sequence, and thereby producing 2-3 orders of magnitude more transmembrane protein in a mammalian cell as compared to standard methods. The method provided can be accomplished by, for example, by optimization of various steps including the contacting and introducing steps.

In one embodiment, there is provided an in vivo method for producing about 2-3 orders of magnitude more cytosolic protein in a mammalian cell as compared to standard methods by contacting a nucleic acid sequence encoding the transmembrane protein and operably linked to regulatory elements with a skeletal muscle cell of a subject, and introducing the nucleic acid sequence into the cell using electroporation, wherein expression of the transmembrane protein is by endogenous translation of the nucleic acid sequence, and thereby producing 2-3 orders of magnitude more cytosolic protein in a mammalian cell as compared to standard methods. The method provided can be accomplished by, for example, by optimization of various steps including the contacting and introducing steps.

In another embodiment of the invention, there is provided an in vivo method for expressing about 2-3 orders of magnitude more protein in a skeletal muscle cell from a polynucleotide encoding the protein, by contacting-the skeletal muscle cell with a tissue permeability enhancing agent; contacting the skeletal muscle cell with a recombinant expression vector encoding the transmembrane protein operably linked to a suitable promoter; and applying an electrical stimulus to the muscle cell. The method provided can further comprise a fluorescent and histidine tag. The recombinant proteins expressed by the methods herein can be, for example, a transmembrane protein, a receptor transmembrane protein, a channel/pump, or a soluble protein.

Yet, in another embodiment of the invention, there is provided a mammalian skeletal muscle cell that produces large quantities of a recombinant transmembrane and/or cytosolic and soluble protein. The muscle cells provided herein produce proteins of up to about 1 mg/gram of tissue.

### BRIEF DESCRIPTION OF THE DRAWINGS

FIG. 1 shows stacked two-photon laser scanning microscopy (TPLSM) images of recombinantly expressed pEGFP-N2 in skeletal muscle cells: Panel A1 is a an image of an FDB muscle dissected 5 days after transfection with pEGFP illuminated with white light; Panel A2 is an image from the same muscle as FIG. A1 illuminated with monochromatic blue light (480 nm) and fluorescence filtered with a 550 nm long pass filter; Panel B1 is an image of an FDB muscle dissected 12 hours after transfection and rendered in 256 intensity levels of green, spanning a fluorescence scale of 0-1,500 arbitrary units (AU) in the TPLSM; Panel B2 is an FDB muscle dissected 5 days after transfection. The 256 intensity levels of green span a fluorescence scale of 0-65,536 AU in the TPLSM; and Panel C is a single TPLSM section image through a bundle of fibers from the same muscle as shown in panel B2.

FIG. 2 shows various physiobiochemical analysis of recombinantly expressed pEGFP-N2 in skeletal muscle cells: Panel A shows an SDS-PAGE of supernatant fractions obtained from a control (lane 1) and a transfected (lane 2) FDB muscle; Panel B shows a Western Blot analysis of a replica of the SDS-PAGE shown in panel A indicating a protein band having about the same molecular weight; Panel C is a graph showing the fluorescence emission spectra of 1:20 dilution of the supernatant obtained from a pEGFP transfected FDB muscle (trace a), a control muscle (trace c), and 10 μg/ml commercial EGFP (trace b); and Panel D is a graph showing traces a and b of panel C normalized to their respective peaks at 508 nm and superimposed.

FIG. 3 shows the time course of expression of recombinant pEGFP-N2 in lower limb muscles: Panel A is a SDS-PAGE of supernatant fractions obtained from lower limb muscles transfected with pEGFP-N2 taken after 0.5, 1, 2, 4, 8, 16, 24 and 31 days from transfection (lanes 1-8, respectively); and Panel B is a Western Blot of a replica of the gel shown in panel A.

FIG. 4 shows a graph of the time course of expression of recombinant pEGFP-N2 protein yield in lower limb muscle (mg of EGFP per g wet weight of lower limb muscle tissue), plotted as a function of the time after muscle electroporation. Both axes are displayed in logarithmic scales.

FIG. 5 shows stacked TPLSM images (low and high magnification) of the expression of recombinant EGFP-β1α-DHPR.

FIG. 6 is a graph showing the quantitation of recombinantly expressed pEGFP-β1a-DHPR protein.

FIG. 7 is a Western blot of muscle cell fractions (crude homogenate, supernatant fraction and microsomal fraction) post transfected with pT7-β1a-DHPR, and probed with an anti-T7 monoclonal antibody.

FIG. 8 is a Western blot of muscle cell soluble (S) and membrane (M) fractions post transfected with pECFP-β1a-DHPR and probed with an anti-YFP antibody.

FIG. 9 is an emission spectra graph of recombinant ECFP-β1α-DHPR from post transfected calf muscle cells.

FIG. 10 is a transmission electron microscopic (TEM) image of muscle fibers post transfected with pGFP-α1S-DHPR in muscle fibers.

FIG. 11 shows stacked TPLSM images (low and high magnification) of the expression of recombinant EGFP-α1S-DHPR in muscle fibers.

FIG. 12 shows stacked TPLSM images (low and high magnification) of the expression of recombinant EGFP-Shaker channel proteins in muscle fibers.

FIG. 13 shows TEM images of the expression of recombinant EGFP-Shaker channel proteins in muscle fibers.

FIG. 14 shows stacked TPLSM images (low and high magnification) of the expression of recombinant YFP-RyR1 proteins in muscle fibers.

FIG. 15 are graphs showing action potential and Ca⁺⁺ recordings in muscle cells transfected with pECFP-β1a-DHPR and pEYFP-β1a-DHPR.

FIG. 16 is a stained SDS-PAGE showing the purified recombinantly expressed T7-β1a-DHPR protein.

### DETAILED DESCRIPTION OF THE INVENTION

The present invention provides methods for producing recombinant proteins in mammalian skeletal muscles by in vivo transfection of DNA.

The present invention is based on the discovery that skeletal muscle fibers can be used to produce large quantities of heterologous proteins, for example, transmembrane and eukaryotic cytosolic proteins. Skeletal muscle fibers are ideal for expression for a variety of reasons, including: muscle fibers have a large ratio of external and internal membrane compartments relative to their volume; muscle fibers represent a large proportion of the body mass; muscle fibers are easily accessible for transfection procedures; muscle fibers are composed of large syncytial post-mitotic cells with large capacity to synthesize proteins; and are multinucleated cells with the potential to synthesize large quantities of protein while maintaining a physiological protein-to-lipid ratio. Lu, Q. L., G. Bou-Gharios, and T. A. Partridge, “Non-viral gene delivery in skeletal muscle: a protein factory,” Gene Ther., 2003, 10(2): p. 131-42; and Needham, D. M., Machina Carnis (1971), The biochemistry of muscular contraction in its historical development, London: Cambridge University Press.

The present invention describes polynucleotides encoding certain polypeptides, including: the α subunit of the skeletal muscle DHPR (α1S-DHPR), a voltage dependent Ca²⁺ channel (VDCC) endogenously expressed in the T-tubule membranes of skeletal muscle fibers and an important component in the excitation-contraction (EC) coupling process; the voltage-dependent Shaker K channel, which is not expressed endogenously in skeletal muscle, but is a heavily studied ion channel; and the mammalian ryanodine receptor (RyR1), a Ca²⁺ release channel which is endogenously present in the sarcoplasmic reticulum (SR) membranes of skeletal muscle. Hence, the invention herein demonstrates that both transmembrane and cytosolic (soluble) proteins can be produced in large quantities by the methods described herein.

Yet, the invention is not limited to the expression of the desired proteins herein described, rather polynucleotides encoding other proteins or peptides are fully encompassed by the invention. The desired polypeptides or polypeptides of interest can be a native polypeptide, a homolog thereof, or a substantially related polypeptide but for conservative variations or mutations, such polypeptides are encompassed by the invention. Hence, the invention encompasses various “mutated DNA sequences”. “Mutated DNA sequences” as used herein refer to as any cellular endogenous genomic DNA sequence which has undergone alteration. It is generally anticipated that mutated DNA sequences will be generated upon site-specific homologous recombination or by any other method well known in the art (e.g. PCR, enzyme mediated and the like). Mutated DNA sequences provide advantages when determining the function and integrity of the native protein, as compared to the mutated protein.

Other mutations contemplated, are conservative variations, which denote the replacement of an amino acid residue by another, biologically similar residue. Examples of conservative variations include the substitution of one hydrophobic residue such as isoleucine, valine, leucine or methionine for another or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine. As used herein, the term “conservative variation” also includes the use of a substituted amino acid in place of an unsubstituted parent amino acid provided that antibodies raised to the substituted polypeptide also immunoreact with the unsubstituted polypeptide.

In addition, the present invention also contemplates various modifications and substitutions of the desired recombinant proteins, which are not limited to replacement of amino acids. For a variety of purposes, such as increased stability, solubility, or configuration concerns, one skilled in the art will recognize the need to introduce, (by deletion, replacement, or addition) other modifications. Examples of such other modifications include incorporation of rare amino acids, dextra-amino acids, glycosylation sites, cytosine for specific disulfide bridge formation. The modified peptides can be chemically synthesized, or the isolated gene can be site-directed mutagenized, or a synthetic gene can be synthesized and expressed in bacteria, yeast, baculovirus, and tissue culture and so on.

Once a polypeptide or peptide agent has been identified, peptide analogs are commonly made. Peptide analogs are commonly used in the pharmaceutical industry as non-peptide drugs with properties analogous to those of the template peptide. These types of non-peptide compound are termed “peptide mimetics” or “peptidomimetics”. See Fauchere, 1986, Adv. Drug Res. 15:29; Veber & Freidinger, 1985, TINS p. 392; and Evans et al., 1987, J. Med. Chem. 30:1229, which are incorporated herein by reference for any purpose. Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH2— NH—, —CH2—S—, —CH2—CH2—, —CH═CH— (cis and trans), —COCH2—, —CH(OH)CH2—, and —CH2SO—, by methods well known in the art. Systematic substitution of one or more amino acids of a consensus sequence with a D-amino acid of the same type (e.g., D-lysine in place of L-lysine) may be used in certain embodiments to generate more stable peptides. In addition, constrained peptides comprising a consensus sequence or a substantially identical consensus sequence variation may be generated by methods known in the art (Rizo & Gierasch, 1992, Ann. Rev. Biochem. 61:387, incorporated herein by reference for any purpose); for example, by adding internal cysteine residues capable of forming intramolecular disulfide bridges which cyclize the peptide.

The invention discloses that the expressed β1a-DHPR protein likely interacts and binds to α1S-DHPR. Hence, expression of recombinant proteins may specifically interact with other recombinant heterologous proteins or endogenously proteins or agents (e.g., a recombinant proteins which binds to an endogenous receptor in the transfected cell), which directly and/or indirectly modulate the expression or activity of the recombinantly expressed protein. Examples of agents include, but are not limited to, drugs or therapeutic compounds, toxins, cytokines, and bioactive peptides.

Once the desired protein has been identified, the nucleotide sequence encoding the protein or agent (e.g., polypeptide, peptide, antibody, and functional fragments thereof), may be inserted into a recombinant expression vector. A recombinant expression vector generally refers to a plasmid, virus or other vehicle known in the art that has been manipulated by insertion or incorporation of a nucleic acid sequences. For example, a recombinant expression vector of the invention includes a polynucleotide sequence encoding a EGFP, ECFP, EYFP, β1a-HDPR, α1S-DHPR, RyR1 and Shaker K channels polypeptide or having EGFP, ECFP, EYFP, β1a-HDPR, α1S-DHPR, RyR1 activity or a fragment thereof or encoding an EGFP, ECFP, EYFP, β1a-DHPR, α1S-DHPR, RyR1 fusion product or fragment thereof. The expression vector typically contains an origin of replication, a promoter, as well as specific genes which allow phenotypic selection of the transformed cells. Vectors suitable for use in the invention include, but are not limited to the T7-based expression vector for expression in bacteria (Rosenberg, et al., Gene 56:125, 1987), the pMSXND expression vector for expression in mammalian cells (Lee and Nathans, J. Biol. Chem. 263:3521, 1988), baculovirus-derived vectors for expression in insect cells, cauliflower mosaic virus, CaMV; tobacco mosaic virus, TMV.

Although the invention describes various commercial vectors (e.g., pEGFP, pECFP, pEYFP and the like, from Clontech), other vectors having similar function are encompassed by the invention. For example, nucleotide sequences encoding EGFP, ECFP, EYFP, β1a-DHPR, α1S-DHPR, RyR1 and Shaker K channel proteins, and functional fragments thereof, are inserted or incorporated into E. coli vectors. However, other vectors from other systems can also be used and are contemplated by the invention. For example, in yeast, a number of vectors containing constitutive or inducible promoters may be used. For a review see, Current Protocols in Molecular Biology, Vol. 2, Ed. Ausubel, et al., Greene Publish. Assoc. & Wiley Interscience, Ch. 13, 1988; Grant, et al., “Expression and Secretion Vectors for Yeast,” in Methods in Enzymology, Eds. Wu & Grossman, 1987, Acad. Press, N.Y., Vol. 153, pp. 516-544, 1987; Glover, DNA Cloning, Vol. 11, IRL Press, Wash., D.C., Ch. 3, 1986; and Bitter, “Heterologous Gene Expression in Yeast,” Methods in Enzymology, Eds. Berger & Kimmel, Acad. Press, N.Y., Vol. 152, pp. 673-684, 1987; and The Molecular Biology of the Yeast Saccharomyces, Eds. Strathern et al., Cold Spring Harbor Press, Vols. I and II, 1982. A constitutive yeast promoter such as ADH or LEU2 or an inducible promoter such as GAL may be used (“Cloning in Yeast,” Ch. 3, R. Rothstein In: DNA Cloning Vol. 11, A Practical Approach, Ed. DM Glover, IRL Press, Wash., D.C., 1986). Alternatively, vectors may be used which promote integration of foreign DNA sequences into the yeast chromosome.

The invention describes a commercially available bacterial plasmid expression vector, however, if a viral expression vector has similar transfection efficacy and recombinant protein expression efficiency, then viral expression vectors can be particularly useful. For example, for introducing a polynucleotide encoding a chimeric EGFP, ECFP and/or EYFP into a cell, since viral vectors can infect host cells with relatively high efficiency and can infect specific cell types. For example, a polynucleotide of the invention can be cloned into a baculovirus vector, which then can be used to infect an insect host cell, thereby providing a means to produce large amounts of the encoded chimeric polypeptide. In addition, the viral vector can be derived from a virus that infects vertebrate host cells, particularly mammalian host cells. Viral vectors can be particularly useful for introducing a polynucleotide encoding a chimeric EGFP, ECFP and/or EYFP into a mammalian cell, wherein, upon expression of the chimeric EGFP, ECFP and/or EYFP. Viral vectors have been developed for use in mammalian systems and include, for example, retroviral vectors, other lentivirus vectors such as those based on the human immunodeficiency virus (HIV), adenovirus vectors, adeno-associated virus vectors, herpesvirus vectors, vaccinia virus vectors, and the like (see Miller and Rosman, BioTechniques 7:980 990 (1992); Anderson et al., Nature 392:25-30 Suppl. (1998); Verma and Somia, Nature 389:239-242 (1997); Wilson, New Engl. J. Med. 334:1185-1187 (1996), each of which is incorporated herein by reference).

Also, the vectors described in the invention are capable of producing and expressing the desired proteins. Hence nucleotide sequences of the invention may also be inserted into an expression system which expresses EGFP, ECFP, EYFP, β1a-DHPR, α1S-DHPR, RyR1 and Shaker K channel proteins, and functional fragments thereof, for example, in an insect system (e.g., Drosophilia). In one such system, Autographa californica nuclear polyhedrosis virus (AcNPV) is used as a vector to express foreign or mutated polynucleotide sequences. The virus grows in Spodoptera frugiperda cells. The sequence encoding a protein of the invention may be cloned into non-essential regions (for example, the polyhedrin gene) of the virus and placed under control of an AcNPV promoter (for example the polyhedrin promoter). Successful insertion of the sequences coding for a protein of the invention will result in inactivation of the polyhedrin gene and production of non-occluded recombinant virus (i.e., virus lacking the proteinaceous coat coded for by the polyhedrin gene). These recombinant viruses are then used to infect S. frugiperda cells in which the inserted gene is expressed, see Smith, et al., J. Viol. 46:584, 1983; Smith, U.S. Pat. No. 4,215,051.

Typically, a vector or plasmid is “operatively linked” to appropriate regulatory elements, including, if desired, a tissue specific promoter or enhancer. The plasmids used in the invention have the constitutive mammalian promoters CMV (cytomegalovirus) (e.g., pEGFP from Clontech) or SV40 (simian virus 40) (e.g., pGFP-α1S-DHPR); however, other commercial and proprietary promoters are encompassed by the invention. An expression vector (or the polynucleotide) generally contains or encodes a promoter sequence, which can provide constitutive or, if desired, inducible or tissue specific or developmental stage specific expression of the encoding polynucleotide, a poly A recognition sequence, and a ribosome recognition site or internal ribosome entry site, or other regulatory elements such as an enhancer, which can be tissue specific. For example, a tetracycline (tet) inducible promoter is an example of a promoter that can be useful for driving expression of a polynucleotide, wherein, upon administration of tetracycline, or a tetracycline analog, to a subject containing a polynucleotide operatively linked to a tet inducible promoter, expression of the encoded polypeptide is induced.

The polynucleotides of the invention can also be operatively linked to tissue specific regulatory element, for example, a muscle cell, neuronal cell specific regulatory element, such that expression of an encoded peptide is restricted to neuronal cells in an individual, or to neuronal cells in a mixed population of cells in culture, for example, an organ culture.

As used herein, the term “operatively linked” or “operatively associated” means that two or more molecules are positioned with respect to each other such that they act as a single unit and affect a function attributable to one or both molecules or a combination thereof. Thus, the above described polynucleotides encoding for various polypeptides are typically “operatively linked” or “operatively associated” with other polynucleotides encoding for other proteins or having other functions. For example, a polynucleotide sequence encoding a soluble β1a-DHPR polypeptide can be operatively linked to a regulatory element, in which case the regulatory element confers its regulatory effect on the polynucleotide similarly to the way in which the regulatory element would affect a polynucleotide sequence with which it normally is associated with in a cell. A first polynucleotide coding sequence also can be operatively linked to a second (or more) coding sequence such that a chimeric polypeptide can be expressed from the operatively linked coding sequences. The chimeric polypeptide can be a fusion polypeptide, in which the two (or more) encoded peptides are translated into a single polypeptide, i.e., are covalently bound through a peptide bond; or can be translated as two discrete peptides that, upon translation, can operatively associate with each other to form a stable complex.

The present invention describes a polynucleotide molecule encoding the desired protein and also operatively linked to at least a fluorescent protein tag or markers (e.g. EGFP, EGFP and EYFP). The fluorescent markers defined herein were used as a means to monitor and visualize the localization of the recombinant proteins present within cells. The fluorescent markers or “molecular beacons” of the invention (e.g., EGFP, ECFP and/or EYFP) were used to test the efficacy of the in vivo transfection method, however, other markers are encompassed by the invention. A number of selective agents can be utilized for the detection of a marker presence within cells, so long as they do not require the addition of agents for the identification of marker presence are considered functional if they allow for the isolation of cells containing said selectable marker from cells which contain different selectable markers or no selectable marker. Some examples include, but are not limited to, the fluorescent proteins GFP, CFP, YFP, RFP, dsRED and HcRED, also listed in the Table below.

TABLE 1
Selectable Markers Utilized in Gene Targeting Vectors

| Positive | | Method for |
|---|---|---|
| Marker | Selection Agent | Detection |
| GFP | UV Light | Fluorescence |
| CFP | UV Light | Fluorescence |
| YFP | UV Light | Fluorescence |
| RFP | UV Light | Fluorescence |
| dsRED | UV Light | Fluorescence |
| HcRED | UV Light | Fluorescence |

In addition, the invention also describes a polynucleotide encoding the desired protein is operatively linked to a carboxy-terminal tag, for example, a His-6 tag or the like, which can facilitate identification of expression of the polypeptide in the target cell. Importantly, His tagging provides the basis for the purification and eventual crystallization of the heterologously expressed transgenic protein. Vectors encoding for proteins with multi-histidine affinity tags (6His or 8His) are also disclosed. In such an instance, a polyhistidine tag peptide such as His-6 can be detected using a divalent cation such as nickel ion, cobalt ion, or the like. However, additional peptide tags include, for example, a FLAG epitope, which can be detected using an anti-FLAG antibody (see, for example, Hopp et al., BioTechnology 6:1204 (1988); U.S. Pat. No. 5,011,912, each of which is incorporated herein by reference); a c-myc epitope, which can be detected using an antibody specific for the epitope; biotin, which can be detected using streptavidin or avidin; and glutathione S transferase, which can be detected using glutathione. Such tags can provide the additional advantage that they can facilitate isolation of the operatively linked polypeptide or peptide agent, for example, where it is desired to obtain, for example, a substantially purified soluble EGFP, ECFP, EYFP and β1a-DHPR polypeptide.

The length of the vector will vary depending upon the choice of positive selectable markers, the choice of nucleotide sequences which are transcribed but do not code for a functional protein product, the presence or absence of promoters capable of driving the expression of the positive selectable marker encoded by the second DNA sequence, the length of the first and third DNA sequences required for appropriate homologous recombination, the size of the base vector and the choices for selection of the plasmid vector in bacteria such as ampicillin resistance and the size of the origin of replication for the plasmid backbone. It is reasonably estimated, however, based upon the sizes of known plasmids and positive selectable markers, that the entire vector will be at least several kilobase pairs in length.

Hence, the methods herein describe a combination of proteins operatively linked, or a chimeric polypeptide. A chimeric polypeptide generally demonstrates some or all of the characteristics of each of its peptide components. As such, a chimeric polypeptide can be particularly useful in performing methods of the invention, as disclosed herein. For example, a method of the invention can be practiced by introducing ex vivo into cells of a subject to be treated, or into cells that are haplotype matched to the subject, a polynucleotide encoding soluble EGFP, ECFP, EYFP, β1a-DHPR, α1S-DHPR, RyR1 and Shaker K channel polypeptides operatively linked to a signal peptide that directs secretion or extrusion of the chimeric polypeptide from the cell. The cell then can be administered to the subject, wherein, upon expression of the chimeric polypeptide, the signal peptide directs secretion or extrusion of the polypeptide from the cell and the soluble EGFP, ECFP, EYFP, β1a-DHPR, a1S-DHPR, RyR1 and Shaker channel polypeptide component of the chimeric polypeptide can effect the excitatory, activating or inhibitory action upon contact with another target cell or target protein or agent.

A chimeric polypeptide also can include a cell compartmentalization domain. Cell compartmentalization domains are well known and include, for example, a plasma membrane localization domain, a nuclear localization signal, a mitochondrial membrane localization signal, an endoplasmic reticulum localization signal, or the like (see, for example, Hancock et al., EMBO J. 10:4033-4039, 1991; Buss et al., Mol. Cell. Biol. 8:3960-3963, 1988; U.S. Pat. No. 5,776,689 each of which is incorporated herein by reference). Such a domain can be useful to target a polypeptide agent to a particular compartment in the cell, or, as discussed above, to target the polypeptide for secretion from a cell.

The invention also describes vectors used to transform a host cell, e.g. skeletal muscle fibers. As used herein, the term “transform” or “transformation” or “transfect” or equivalents thereof, refers to a permanent or transient genetic change induced in a cell following incorporation of new DNA (i.e., DNA exogenous to the cell). Where the cell is a mammalian cell, a permanent genetic change is generally achieved by introduction of the DNA into the genome of the cell.

Further, a polynucleotide of the invention, or a vector containing the polynucleotide can be contained in a cell, for example, a “host cell”, a “transformed cell” or “transfected cell” generally refers to a cell (e.g., prokaryotic or eukaryotic) into which (or into an ancestor of which) has been introduced, by means of recombinant DNA techniques, a DNA molecule encoding ,for example, EGFP, ECFP, EYFP, β1a-DHPR, a1S-DHPR, RyR1 and Shaker channel proteins, or functional fragment thereof. A host cell allows propagation of a vector containing the polynucleotide, or a helper cell, which allows packaging of a viral vector containing the polynucleotide. The polynucleotide can be transiently contained in the cell, or can be stably maintained due, for example, to integration into the cell genome.

Transformation of a host cell with recombinant DNA may be carried out according to the methods described herein, or by conventional techniques as are well known to those skilled in the art. Where the host is either prokaryotic or eukaryotic. When the host is a eukaryote, methods of transfection or transformation with DNA include calcium phosphate co-precipitates, conventional mechanical procedures such as microinjection, electroporation, insertion of a plasmid encased in liposomes, or virus vectors, as well as others known in the art, may be used. Eukaryotic cells can also be co-transfected with DNA sequences encoding an EGFP, ECFP, EYFP, β1a-DHPR, a1S-DHPR and Shaker K channel proteins, or functional fragments thereof, and a second foreign DNA molecule encoding any number of desired polypeptides, for example, a selectable marker. Typically, a eukaryotic host will be utilized as the host cell. The eukaryotic cell of the invention is a muscle cell, however, other hosts including a yeast cell (e.g., Saccharomyces cerevisiae), an insect cell (e.g., Drosophila sp.) or may be a mammalian cell, including a human cell.

The invention describes transfection of a mammalian expression system, which allows for post-translational modifications of expressed mammalian proteins to occur. However, other eukaryotic expression systems, which possess the cellular machinery for processing of the primary transcript, glycosylation, phosphorylation, and, advantageously secretion of the gene product can also be used and is contemplated. Such host cell lines may include, but are not limited to, CHO, VERO, BHK, HeLa, COS, MDCK, Jurkat, HEK-293, and W138.

Still, mammalian cell systems which utilize recombinant viruses or viral elements to direct expression may be engineered. For example, when using adenovirus expression vectors, a polynucleotide encoding EGFP, ECFP, EYFP, β1a-DHPR, a1S-DHPR and Shaker K channel proteins, or functional fragments thereof, may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a encoding an agent which modulates expression or activity of synGAP, or synGAP fusion, or synGAP functional fragments thereof in infected hosts (e.g., see Logan & Shenk, Proc. Natl. Acad. Sci. USA, 81:3655-3659, 1984). Alternatively, the vaccinia virus 7.5K promoter may be used. (e.g., see, Mackett, et al., Proc. Natl. Acad. Sci. USA, 79:7415-7419, 1982; Mackett, et al., J. Virol. 49:857-864, 1984; Panicali, et al., Proc. Natl. Acad. Sci. USA 79:4927-4931, 1982). Of particular interest are vectors based on bovine papilloma virus which have the ability to replicate as extra chromosomal elements (Sarver, et al., Mol. Cell. Biol. 1:486, 1981). Shortly after entry of this DNA into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression a gene encoding EGFP, ECFP, EYFP, β1a-DHPR, a1S-DHPR and Shaker channel proteins, or functional fragments thereof gene in host cells. High level expression may also be achieved using inducible promoters, including, but not limited to, the metallothionine IIA promoter and heat shock promoters.

In one embodiment of the invention, there is provided methods to make large quantities of a recombinant protein via in vivo transfection (e.g., about 4-31 days). The methods described herein show transient expression of recombinant proteins in large quantities. Yet, for longer-term high-yield production of recombinant proteins, stable expression may be required, and such methods are contemplated by the invention. Rather than using expression vectors which contain viral origins of replication, host cells can be transformed with the cDNA encoding the desired protein or agent, for example, EGFP, ECFP, EYFP, β1a-DHPR, α1S-DHPR, RyR1 and Shaker channel proteins, or functional fragments thereof, controlled by appropriate expression control elements (e.g., promoter, enhancer, sequences, transcription terminators, polyadenylation sites, and the like), and a selectable marker. Methods for long-term stable expression of the protein include addition of a selectable marker in the recombinant vector to confer resistance to the selection and allow cells to stably integrate the plasmid into their chromosomes and grow to form foci which in turn can be cloned and expanded into cell lines. For example, following the introduction of foreign DNA, engineered cells may be allowed to grow for 1-2 days in an enriched media, and then are switched to a selective media. A number of selection systems may be used, including, but not limited to, the herpes simplex virus thymidine kinase (Wigler, et al., Cell, 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy, et al., Cell, 22:817, 1980) genes can be employed in tk-, hgprt- or aprt- cells respectively. Also, anti-metabolite resistance can be used as the basis of selection for dhfr, which confers resistance to methotrexate (Wigler, et al., Proc. Natl. Acad. Sci. USA, 77:3567, 1980; O'Hare, et al., Proc. Natl. Acad. Sci. USA, 8:1527, 1981); gpt, which confers resistance to mycophenolic acid (Mulligan & Berg, Proc. Natl. Acad. Sci. USA, 78:2072, 1981; neo, which confers resistance to the aminoglycoside G-418 (Colberre-Garapin, et al., J. Mol. Biol. 150: 1, 1981); and hygro, which confers resistance to hygromycin (Santerre, et al., Gene 30:147, 1984) genes. Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA 85:8047, 1988); and ODC (ornithine decarboxylase) which confers resistance to the ornithine decarboxylase inhibitor, 2-(difluoromethyl)-DL-ornithine, DFMO (McConlogue L., In: Current Communications in Molecular Biology, Cold Spring Harbor Laboratory, ed., 1987).

The invention investigates the role of key proteins in the EC coupling process of skeletal muscle fibers. During EC coupling, the action potential elicited by electrical stimulation of skeletal muscle fiber spreads throughout the membranes of the transverse tubules (T-tubules; a membrane compartment continuous with the surface membrane) and leads to the release of Ca²⁺ ions from the SR (an intracellular membrane compartment). The proteins most likely involved in the transduction between the depolarization of the T-tubules and the release of Ca²⁺ from the SR are the α1S-DHPR, the pore-forming subunit of the VDCC, which probably acts as a voltage sensor in the T-tubules, and the ryanodine receptor of the SR membrane (RyR1, the Ca²⁺ release channel in adult mammalian cells). In order to gain insight about molecular interactions that have been hypothesized to occur between these two membrane proteins, the inventors considered the possibility of expressing fluorescent recombinant forms of these proteins in murine adult skeletal muscles in order to perform fluorescence resonance energy transfer (FRET) measurements.

The invention describes in vivo transfection of skeletal muscle cells. There are several features that confer mammalian skeletal muscle the capability to behave as an ideal transgenic protein factory and some of them may underlie the pattern of extracellular accumulation of membrane proteins described above. Firstly, skeletal muscle is easily accessible for pDNA delivery to hundreds of elongated cylindrical cells using a relatively non-invasive protocol that consists in subcutaneous injection of the plasmids and in vivo electrical stimulation. Skeletal muscle fibers are fully differentiated cells with post-mitotic nuclei ideally suited for the continuous production of transgenic proteins. Moreover, the syncytial nature of muscle fibers (each muscle fiber is a multinucleated cell with about 100 nuclei [for a typical FDB fiber] evenly distributed nuclei along the perimeter of the fiber, underneath the sarcolemma) provides a mechanism for internal dispersal of plasmids from a limited site of penetration to a large number of neighboring nuclei within the fiber. It has been suggested that such dispersion within transfected fibers may well be one of the reasons behind the efficient expression of transgenic proteins in muscle. Interestingly, even when muscle fibers are damaged (a possibility considering the about 100 V/cm utilized for in vivo electroporation of the cells) only short segments of individual fibers undergo degeneration, and the nuclei of surviving segments seem to remain viable. In sum, the muscle cell is designed for the continuous synthesis of the large number of proteins necessary for its normal function: to contract in response to electrical activation. A large proportion of these are membrane proteins, responsible for the propagation of the action potential and EC coupling during repetitive stimulation. In addition to this protein synthesis capability, the muscle fiber is the specialized cell with the most sophisticated organization of subcellular membrane compartments: a highly developed network of T-tubules which are open to the extracellular space and represent a membrane area of approximately 10-fold that of the surface membrane, and the SR system represents an intracellular membrane compartment with approximately 100-fold the surface area of the muscle fiber. From these considerations we can conclude that from all the cell types in biology, the muscle fiber is probably the best suited not only to synthesize large quantities of membrane proteins, but also to potentially accommodate them in multiple membrane compartments.

Further the invention describes pDNA compositions and solutions used for in vivo transfection. The pDNA solutions for electroporation of the present invention can be made into a preparation form suitable for physicochemical properties of the active ingredients such as solution, emulsion, semisolid and solid by treating the aforementioned essential ingredients, preferred ingredients, arbitrary ingredients and active ingredients according to a usual method and used for percutaneous administration of the active ingredients together with a device for electroporation. Among these, examples of the preferred pharmaceutical preparation include aqueous preparations, and aqueous solution preparations, aqueous gel preparations, emulsion preparations and so forth are particularly preferred. The composition for electroporation of the present invention is a composition containing alkaline earth metal ions and a carrier for electroporation. The carrier for electroppration is a carrier for formulating such preparations for electroporation as described above, and particularly preferred examples thereof include aqueous solvents, gelling agents, emulsifiers and so forth.

Although the invention describes in vivo transfection methods to deliver the pDNA solutions into the host cell (e.g., skeletal muscle fibers), other targeted delivery systems for polynucleotides are contemplated by the invention. For example, delivery can occur my means of a colloidal dispersion system. Colloidal dispersion systems include macromolecule complexes, nanocapsules, microspheres, beads, and lipid-based systems including oil-in-water emulsions, micelles, mixed micelles, and liposomes. The colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988).

The composition of the liposome is usually a combination of phospholipids, particularly high-phase-transition-temperature phospholipids, usually in combination with esterols, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidyl-glycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes has been classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system (e.g., surface of skeletal muscle cells or mass) may be modified in a variety of ways. For example, the invention describes various muscle groups in mammals because they are accessible and easy to work with. However, in the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. In general, the compounds bound to the surface of the targeted delivery system will be ligands and receptors which will allow the targeted delivery system to find and “home in” on the desired cells. A ligand may be any compound of interest which will bind to another compound, such as a receptor.

The composition of the present invention is a composition for external use, since it is characterized by being used for electroporation. The compositions for external use may be cosmetic compositions or pharmaceutical compositions. However, pharmaceutical compositions are particularly preferred, since they can fully exhibit the effect by their characteristic of significantly promoting percutaneous absorption.

Nucleic acid containing solutions and/or compositions of the invention, for in vivo transfect ions into mammalian expression system (i.e. skeletal muscle fibers), are directly injected. Generally, compositions of the invention are typically administered parenterally (e.g., by injection or by gradual perfusion over time), enterically, by injection (e.g., intravenously, intraperitoneally, intramuscularly, subcutaneously, intracavity, or transdermal), rapid infusion, nasopharyngeal absorption, dermal absorption, rectally and orally. As used herein, the term “administering” or “contacting” is accomplished by any means known to the skilled artisan. Pharmaceutically acceptable carrier preparations for parenteral administration include sterile or aqueous or non-aqueous solutions, suspensions, and emulsions. Examples of non-aqueous solvents are propylene glycol, polyethylene glycol, vegetable oils such as olive oil, and injectable organic esters such as ethyl oleate. Carriers for occlusive dressings can be used to increase skin permeability and enhance antigen absorption. Liquid dosage forms for oral administration may generally comprise a liposome solution containing the liquid dosage form. Suitable solid or liquid pharmaceutical preparation forms are, for example, granules, powders, tablets, coated tablets, (micro)capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners and elixirs containing inert diluents commonly used in the art, such as purified water.

The invention describes in vivo transfected muscles capable of very rapid an efficient synthesis of transgenic proteins. The proteins are properly folded (e.g., in the case of membrane channels, they are probably targeted to the SR, T-tubule and surface membrane) and retain functional activity (e.g., action potentials and Ca++ release in muscle contraction). These studies allow comparison of muscle as an expression system with the most heavily used bacterial expression systems. Bacterial expression systems have the major limitation that many full-length eukaryotic proteins cannot be expressed correctly, as is the case of the β1a-DHPR subunit, probably due to the inability of the bacterial cells to provide adequate supporting machinery for their synthesis, and folding.

Studies of eukaryotic proteins are advantageous of their prokaryotic counterparts. MacKinnon and Doyle identified prokaryotic channels with properties comparable to those of well-characterized eukaryotic channels that can be readily synthesized by bacterial cells. MacKinnon's laboratory crystallized and determined, by X-ray analysis, the atomic structure of many prokaryotic channels, giving key insights into the permeability mechanisms of several types of ionic channels. Nevertheless, there are important properties of eukaryotic voltage-gated channels that are not necessarily emulated by their prokaryotic counterparts. A case in fact is that the voltage-gating mechanisms proposed by MacKinnon and collaborators for a voltage-dependent prokaryotic K channel (KvAP) may not conform with a bulk of our knowledge about eukaryotic K channels (including the Shaker K channel), currently giving rise to an interesting controversy. Furthermore, other ionic channels such as the skeletal muscle DHPR and the RyR1 channels have structural features (revealed with high resolution cryoelectron microscopy) that clearly depart from those learned from prokaryotic channels. All these considerations can be extended to other membrane proteins such as ion transporters, whose structure has been successfully investigated only in homologous prokaryotic transporters.

Another advantage of devising a eukaryotic expression (or over expression) system able to express transgenic proteins, compared to endogenous protein expression within the cell, is that transgenic proteins encoded by pDNA can be suitably tagged with amino acid sequences conferring specific properties. As discussed above, the possibility to express EGFP, ECFP, EYFP tagged proteins has been crucial to monitor the widely different expression pattern of cytosolic versus membrane proteins. Furthermore, fluorescent and 4 His, 6 His, 8 His or more tagging provide excellent tools for the quantitation and purification of proteins and, provided that the tagged proteins are carefully engineered not to interfere with their functions, there is little concern for their usefulness.

The usefulness of the findings of the present invention for scientific purposes in biomedical research and for biotechnological applications in the future, may include, but is not limited to: providing a straightforward methodology to attain the highly elusive goal of synthesizing large quantities of eukaryotic transmembrane proteins; serve as a model on how nature stabilizes membrane proteins; manufacture of artificial conductive elements for biomedical applications; efficient production of antibodies against transmembrane proteins, an effort dwarfed so far by the inability to express in vivo large quantities of transgenic, or chimeric, eukaryotic membrane proteins; implications towards the implementation of new anti-cancer strategies; and the ability to insert large quantities of functional ion channels into membranes of muscle cells, thus opening the door for future correction of muscle channelopathies. The methods of the present invention could provide enough membrane protein to fulfill these needs.

Also, in addition to primates, such as humans, a variety of other mammals can be treated according to the method of the present invention. For instance, mammals including, but not limited to, cows, pigs, sheep, goats, horses, dogs, cats, guinea pigs, rats, or other bovine, porcine, ovine, equine, canine, feline, rodent or murine species can be treated. However, the method can also be practiced in other species, such as avian species (e.g., chickens).

The following examples are intended to illustrate but not limit the invention.

### Materials & Methods

### Animal Model

Young male C57BL mice about 1.5 to 3 months of age, weighing about 20-30 grams, were used. Plasmid transfection was performed in either flexor digitorum brevis (FDB) muscles or a group of “lower limb muscles” such as the soleus, tibialis anterior and extensor digitorum muscles. Still other studies were performed using larger muscles, for example, but not limited to, hamstring muscles. Experiments using animals were carried out according to the guidelines laid down by the UCLA Animal Care Committee.

### Plasmids Amplification

DNA plasmids encoding for enhanced green fluorescent proteins (pEGFP-N2), enhanced cyan green fluorescent protein (ECFP), or yellow fluorescent protein (YFP) were obtained from Clontech (BD Biosciences, Mountain View, Calif.). pEGFP, pECFP or pYFP were operably linked to cDNAs encoding various transmembrane and cytosolic proteins and were obtained as gifts (GFP-α1S-DHPR from K. Beam, Colorado State University, Fort Collins, Colo., and M. Grabner, Universitat Inssbruck, Austria; EYFP-RyR1 from P. Allen, Brigham Women's Hospital, Harvard University, Boston, Mass.; and EGFP-Shaker from F. Bezanilla & R. Blunck, UCLA, Los Angeles, Calif.). The plasmids were amplified in OneShot TOP 10 (Clontech) bacteria and isolated using Qiagen Endo-Free Kits (QIAGEN, Valencia, Calif., USA) following the procedures of the manufacturer.

Muscle Transfection with DNA Plasmids

Muscle transfection in anesthetized animals (isoflurane) was achieved by injection with pDNA, followed by in vivo electroporation. The protocols used in the flexor digitorum brevis (FDB) muscles differed slightly from those in “lower limb” muscles. In the case of FDB, 5 μl of 2 mg/ml of hyaluronidase was dissolved in sterile saline and injected subcutaneously into the foot pads of the animal using a 33 gauge needle. Lu, Q. L. (2003), supra; Favre, D., et al. (2000) “Hyaluronidase enhances recombinant adeno-associated virus (rAAV)-mediated gene transfer in the rat skeletal muscle,” Gene Ther., 7(16):1417-20. About an hour later, about 20 μg of pDNA, dissolved in buffer (10 mM TrisCl, pH 8, 1 mM EDTA) solution, at concentrations of about 2-5 μg/μl, was subcutaneously injected in the muscle pad. In larger animals (e.g. rats), the total injected pDNA can be up to about 150 μg or greater. After about 10 min, two electrodes (200 μm gold plated stainless steel needles) were placed subcutaneously close to the proximal and distal tendons of the muscles and electrical pulses were delivered for muscle electroporation.

The amplitude and number of electrical pulses varied depending on several factors such as the pDNA solution, the size of the cDNA insert, the muscle mass, and the size of the animal. For example, in a young mouse FDB muscle, about 20 pulses of 100 V in amplitude, of about 20 ms in duration, were applied at a frequency of 1 Hz. Pulses were generated by a Grass S88 medical stimulator (Grass, Quincy, Mass., USA). For lower limb muscles, the protocol was substantially similar except that the injections of hyaluronidase and pDNA were applied intramuscularly at 3 positions (equidistant locations between the ankle and the knee) of the lower limb muscles. Stimulating electrodes were placed parallel to the leg axis and inserted subcutaneously at both sides of the leg. In general, right muscles were transfected while left muscles were used as contra-lateral controls.

### Two Photon Laser Scanning Confocal Microscopy (TPLSM)

Muscles were dissected from the anesthetized animals and fixed to Sylgard-bottomed Petri dishes and placed on the stage of an upright microscope (Olympus, BX51WI) equipped with an adjustable wavelength Chameleon Ti/Sapphire laser system (Coherent) and a Radiance 2000 Scanning Head (Bio-Rad, UK). The fluorescent proteins (e.g., EGFP, ECFP and EYFP) were excited according to manufacturer recommendations. For example, EGFP was excited at 880 nm and its fluorescence detected through a 495-515/30 dichroic-emission filter combination. Chen, Y. and A. Periasamy (2004) “Characterization of two-photon excitation fluorescence lifetime imaging microscopy for protein localization,” Microsc Res Tech, 63(1):72-80. Low and high magnification TPLSM images were obtained with a 10×, NA 0.45 (Olympus) and a 20×, NA 0.95 (Olympus XLUMPLANFL) lenses. Images were constructed and superimposed from TPLSM sections spaced at about 5 μm for low magnification, and at about 2 μm for high magnification. Images were analyzed using commercial and public domain image analysis software packages (e.g., LaserSharp 2000, Confocal Assistant, and ImageJ).

### Muscle Homogenization and Fractionation

Muscles were blot dried and weighed. Tendons were trimmed and muscles minced into small pieces using razor blades. Homogenization buffer, consisting of 150 mM KCl, 5 mM MgSO₄, 20 mM MOPS (pH 7.00), protease inhibitor cocktail (Sigma) 1:50, 0.1 mM PMSF, was added to the tissue at a ratio of 4 μl/mg. Homogenization was performed with a glass tissue grinder. Supernatant fractions of muscle homogenates were obtained following 2 steps of centrifugation at 1,500 rpm (Eppendorf, 5415C), and one at 20,000 rpm (Beckman Coulter, Avanti J20 XP). Samples of crude, supernatant and microsomal fractions from the muscle cell fiber homogenates were collected.

### Protein Quantitation

Protein concentration was determined using a commercial kit (Quick Start Bradford Dye Reagent, Bio-Rad) and BSA as a standard. Absorbance measurements were done in an HP8453 UV-Visible System (Hewlett-Packard, Palo Alto, USA). Purified bacterial EGFP (rEGFP, Clontech) was utilized to calibrate the concentrations of EGFP in the supernatants. To this end, the fluorescence of solutions at protein concentration ranging from 0.625 to 20 μg/ml was measured using a dual beam spectrofluorimeter (Jobin Yvon Fluorolog, FL3-21, Edison, N.J., USA). Fluorescence values at 508 nm were plotted as a function of the protein concentration and regression lines were fitted to the data. The EGFP concentrations in muscle supernatants were obtained by interpolation.

### SDS-PAGE and Western Blotting

Prior to the analytical characterization of proteins by gel electrophoresis, endogenous muscle soluble proteins were partially removed from supernatant fractions by heat shock treatments that did not affect their EGFP contents. See Bokman, S. H. and W. W. Ward, (1981), “Renaturation of Aequorea green-fluorescent protein,” Biochem Biophys Res Commun, 101(4):1372-80. The supernatant was heated at 70° C. for 5 min and concentrated 3 fold in a refrigerated evaporator (Speed Vac, SVC 100, Savant Instruments). The treated supernatants were mixed 1:1 with sample buffer (62.5 mM TRIS, ph 6.8, 0.1% glycerol, 2% SDS, 5% 2 mercaptoethanol, 0.05% bromophenol blue, 50 mM DTT) and denatured by boiling for 5 min. See Laemmli, U.K. (1970) “Cleavage of structural proteins during the assembly of the head of bacteriophage T4,” Nature, 227(259): p. 680-5. Aliquots of this solution were loaded in duplicated 16% Polyacrylamide SDS gels. Molecular weight markers (SeeBlue Plus2 Pre-Stained Standard) were obtained from Invitrogen (Carlsbad, Calif., USA). Protein bands were stained with Imperial Protein Staining (Pierce, Rockford, Ill., USA).

Methods for transferring the proteins from SDS gels onto nitrocellulose for Western blotting are standard in the art. Towbin, H., T. Staehelin, and J. Gordon (1979), “Electrophoretic transfer of proteins from polyacrylamide gels to nitrocellulose sheets: procedure and some applications,” Proc Natl Acad Sci USA, 76(9):4350-4354. The blots were incubated with primary EGFP/anti-GFP antibody was (Clontech), and a secondary anti-mouse IgG antibody (Santa Cruz Biotechnology, Santa Cruz, Calif., USA). The membranes were incubated with a chemiluminescent substrate (Inmun-Star HRP Chemiluminescent Kit, Bio-Rad) at room temperature for 5 min. Signals were detected with a chemiluminescent imaging system (ChemiDoc System EQ, BioRad) and stored digitally.

### Recombinant Protein Purification

Recombinantly expressed proteins were purified using standard methods in the art, for example, the 6 His-tagged recombinant β1a-DHPR, was purified using a TALON Co²⁺ column (Clontech). However, any method of protein purification using various affinity chromatography, size-exclusion chromatography, ionic-exchange chromatography, and the like is within the scope of the invention. β1a-DHPR was purified to show that the level of recombinant protein production using the methods described herein, produces enough protein for purposes of protein purification. A purified protein is advantageous for various biological uses including performing crystalline structure analysis.

### Functional Assays

Studies of the excitation and contraction coupling of skeletal transfected skeletal muscles were performed. The action potentials and thereby function of the various transfected muscle cells was determined using procedures standard in the art.

All experiments were carried out according to the guidelines laid down by the local Animal Care Committee. Single muscle fibres were enzymatically isolated from flexor digitorum brevis (FDB) and extensor digitorum longus (EDL) muscles dissected from normal (C57BL/I0SnJ) mice (Jackson Laboratories, Me.). Both of these muscles are fast-twitch (type II) fibres. All experiments were done in 8-18 week old normal animals. Mice were anesthetized with halothane (loss of righting reflex) and sacrificed by cervical dislocation. Muscles were removed, stored in cold (5° C.) Tyrode solution (see below) and utilized within 30 minutes.

The digestion and dissociation protocol is as follows: Each muscle was placed in a Sylgard-bottomed Petri dish with its tendons held in place by pins and bathed in 0-Mg²⁺/0-Ca²⁺-Tyrode supplemented with 262 units/ml of collagenase Type IV (Sigma, St. Louis, Mo.) and 0.5 mg/ml of bovine serum albumin. They were incubated for 45 min at 37° C. under mild agitation. Collagenase activity was stopped by washing the muscle with 0-Mg²⁺/0-Ca²⁺-Tyrode at 37° C. The muscle mass was gently sucked in and out of a fire-polished Pasteur pipette until muscle fibres were isolated. For the FDB fibers, the average diameter and length were ˜30 and ˜300 μm, respectively. For the EDL, the average diameter and length were ˜60 μm and ˜6 mm, respectively. This studied showed that immediately following this dissociation, very few of the large number of fibres isolated actively twitched in response to external electrical stimulation when bathed in external solutions containing 2.0 mM [Ca²⁺]. However, after a period of 30 minutes of incubation in L-15 media (Sigma, St. Louis, Mo.) supplemented with 0.1 mg/ml penicillin/streptomycin (Sigma, St. Louis, Mo.) and maintained in an O₂ saturated environment at 25° C., approximately 25% of the fibres responded to external stimulation with vigorous twitches. Only this population of fibres was used.

The electrophysiological method used for enzymatically dissociated EDL fibres was similar to the triple Vaseline gap technique previously described for mechanically dissected frog muscle fibres. The silicone compound (Chemplex 825, NFO Technologies, KS) was used to construct the seals, instead of petroleum jelly compounds, and 1% fetal calf serum (FCS, Irvine Scientific) was added in the external solution to further improve the tolerance of the fibres to Chemplex 825. The fibres were straightened across the gaps, maintained at slack sarcomere length (˜2 μm) and loaded with internal solution (see below) through the cut ends. The holding potential of the central pool was set at −95 mV.

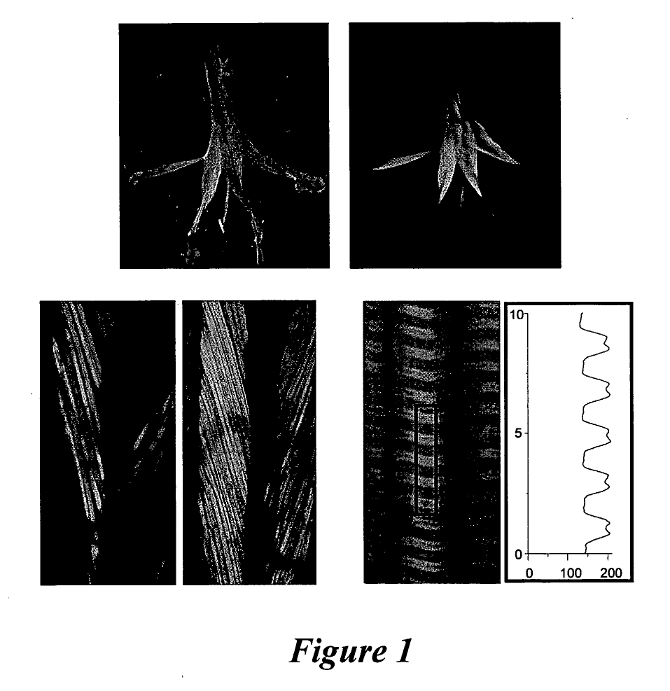

In the case of FDB muscle fibres, they were transferred to an optical chamber containing Tyrode solution and impaled with two micropipettes. These micropipettes were placed ˜150 μm apart in un-stretched fibres with a sarcomere length of 2 μm, as shown in the phase contrast image in FIG. 1A. The first one was a high resistance micropipette (pipette 1, FIG. 1A) which had ˜40 MΩ resistance when filled with 3M KCl and the second one was a low resistance micropipette (pipette 2, FIG. 1A) which had a resistance of ˜30 MΩ when filled with internal solution. Pipette 1 was used to record the transmembrane potential (Vₘ). No current was passed through this electrode. Pipette 2 was used to load the fibre with internal solution, to maintain its resting potential, and to stimulate it. Both micropipettes were connected to a TEV-200A amplifier (Dagan, Minneapolis, Minn.). As expected, the recordings from pipette 2 show a saturating stimulus artifact that prevents the recording of the initial part of the rising phase of the AP. As the artifact could not be eliminated, we included a second microelectrode (pipette 1) to faithfully acquire the AP.

The FDB fibres were loaded passively with the internal solution by a mechanism comparable to that described for patch clamp experiments. In agreement with the theoretical calculations for loading, by 30 min following impalement, the properties of ΔF/F transients (see below) were stable within 8% of each other (standard deviation/mean). In addition, in the presence of at least 5 mM EGTA, fibre movement was arrested by 30 min as judged both by the absence of movement artifacts in the electrical and optical records, as well as by visual inspection of the fibre imaged with 100× magnification on the screen of a monitor. Thus, all optical measurements were initiated 30 min after fibre impalement. After recordings were started, the fluorescence transients were stable, typically for 30 more min and in some cases for as long as 90 more min. In addition, ΔF/F transients measured near pipette 2 and at the ends of the fibre had similar properties. Thus, after the equilibration period, the concentrations of solutes in the fibre are assumed to approximate that of the internal solution in pipette 2, with the caveat that binding to internal constituents of the fibre may prevent the attainment of a true steady state.

[Ca²⁺] was measured using the cell impermeant forms of the Ca²⁺ indicators Oregon Green 488 BAPTA-5N (OGB-5N, Molecular Probes, Eugene, Oreg.) or Oregon Green 488 BAPTA-1 (OGB-1, Molecular Probes, Eugene, Oreg.). The internal solution [OGB-5N] was 500 μM and that of [OGB-1] was 50 μM. The free [Ca²⁺] of the internal solutions were determined by interpolation from the OGB-1 calibration curve. Fluorescence measurements of the internal solution samples containing 20 μM OGB-(with or without EGTA) were made and pCa values were interpolated from the dye calibration curves. In so doing, the free [Ca²⁺] of the internal solution was estimated to be 64±5 nM in the absence of EGTA and 2±1 nM in the presence of 5-10 mM EGTA.

Spatially averaged AP evoked fluorescence transients were recorded with OGB-5N or OGB-1 using either an inverted microscope (FDB experiments) or an upright microscope (EDL experiments). Both optical setups were described previously. The fluorescence transients were normalized to ΔF/F units and characterized according to the following parameters as previously described: (ΔF/F)ₚₑₐₖ; duration, expressed as the full-duration-half maximum (FDHM); and delay to peak (tₚ), measured from the initiation of the rising phase of the transient to (ΔF/F)ₚₑₐₖ. The decay phase of the OGB-5N transients was fitted, using a least-squares fitting routine (Origin 6.1, Microcal, Northhampton, Mass.) to the following biexponential function:

$$y = {A}_{fast} {e}^{\frac{- t}{{τ}_{fast}}} + {A}_{slow} {e}^{\frac{- t}{{τ}_{slow}}} \qquad ( 1 )$$

Where t is the time (in ms) after the peak of the transient, and A_{fast} and A_{slow} are fitted amplitudes of the fast and slow components, the sum of which cannot exceed the (ΔF/F)ₚₑₐₖ.

Assuming that OGB-5N was at equilibrium with the free peak [Ca²⁺] and that the resting fluorescence of the indicator in the fibres is equal to Fₘᵢₙ, the free peak [Ca²⁺] underlying the (ΔF/F)ₚₑₐₖ values of the OGB-5N transients could be estimated according to the following equation:

$$Peak [ {Ca}^{2 +} ] = {K}_{d} \frac{{( \frac{Δ F}{F} )}_{peak}}{( R - 1 ) - {( \frac{Δ F}{F} )}_{peak}}$$

The values for R for OGB-5N were measured in vivo and found to be 11±3 (n=3), comparable to the in vitro condition.

To determine the AP evoked SR Ca²⁺ release flux, OGB-5N ΔF/F transients recorded in the presence of high internal [EGTA] were analyzed using a single compartment kinetic model including EGTA, the Ca²⁺ indicator, and Ca²⁺ as the reactants with rate constants determined in vitro. The model generated ΔF/F transients according to the following equation:

$$\frac{Δ F ( t )}{F} = \frac{[ BCa ( t ) ] - [ B Ca ( 0 ) ]}{\frac{[ {B}_{tot} ]}{R - 1} + [ B Ca ( 0 ) ]}$$

where [BCa(t)] is the time-dependent concentration of the Ca-bound indicator and [Bₜₒₜ] is the total concentration of indicator in the cell. The initial concentration of the Ca-bound indicator ([BCa(0)]) was calculated according the following formula:

$$[ BCa ( 0 ) ] = \frac{[ {B}_{tot} ] \cdot [ {Ca}^{2 +} ( 0 ) ]}{{K}_{d} + [ {Ca}^{2 +} ( 0 ) ]}$$

where [Ca²⁺(0)] is the resting (initial) [Ca²⁺] taken as the value in the internal solution in the presence of 5 mM [EGTA]. The kinetics of the Ca²⁺ release flux (J(t)) was described by the equation:

$$J ( t ) = {J}_{max} \cdot {( 1 - {e}^{\frac{- t}{{τ}_{on}}} )}^{N} \cdot ( {A}_{1} {e}^{\frac{- t}{{τ}_{off 1}}} + {A}_{2} {e}^{\frac{- t}{{τ}_{off 2}}} + {A}_{3} {e}^{\frac{- t}{{τ}_{off 3}}} )$$

here t is time (in ms) beginning at the rising phase of the transient. Jₘₐₓ ( in μ/ms), A₁, A₂ and A₃ (dimensionless), and τₒₙ, τ_{off1}, τ_{off2} and τ_{off3} (in ms), were adjusted until the time course of the simulated ΔF/F transient closely approximated the measured transient.

### Statistics

For statistical analysis of the data, the parameters described above were determined from a minimum of 10 individual AP evoked transients per fibre, separated in time such that the (ΔF/F)ₚₑₐₖ remained within the stability definition above, and averaged to determine a set of mean values for the fibre. The data is presented as mean±standard error of the mean (SEM). An unpaired two-population student T-test assuming unequal variance was used to compare the mean fibre values between mdx and normal mice. P values are given in the text.

### EXAMPLE 1

### In Vivo Expression of Green Fluorescent Protein in Skeletal Muscle

This example demonstrates the efficacy of the in vivo transfection of cytosolic proteins into skeletal muscle.

The muscles chosen for the physiological experiments were the flexor digitorum brevis (FDB) and the soleus, which are typical examples of fast and slow muscles, respectively. At least one advantage of these muscle groups is that plasmid (pDNA) solutions can be injected subcutaneously in the feet pads. Another advantage is that uniform electric fields between two parallel subcutaneous electrodes are attainable with this model.

To determine the efficacy of the in vivo transfection method described herein, plasmids (pEGFP-N2 and pECFP, Clontech) encoding enhanced green fluorescent protein (EGFP), or enhanced cyan fluorescent variant (ECFP) of the Aequorea victoria GFP were used to evaluate the EGFP/ECFP protein expression pattern in the muscle fibers using TPLSM. The efficiency of transfection is evidenced by the presence of a large proportion of fluorescent muscle fibers.

### Result

In vivo electroporation of skeletal muscles with plasmids encoding for EGFP and ECFP (used as test proteins) results in the relatively fast expression of cytosolic GFP proteins as evidenced by the appearance of fluorescence in the muscle fibers within about 6 hours after transfection (data not shown). FIG. 1 (panel B1) shows an image of an FDB muscle dissected about 12 hours after in vivo transfection. The image was obtained by stacking 11 consecutive TPLSM sections, and was rendered in 256 intensity levels of green, spanning a fluorescence scale of 0-1,500 arbitrary units (AU) in the TPLSM. After approximately 48 hours, efficiency of transfection (measured as the % muscle fibers showing significant fluorescence) was greater than 90% (data not shown). At about 48 hours post-transfection, the fluorescent proteins were retained in the cytosol of the muscle fibers, but showed a banded appearance, suggesting slight inhomogeneities in their distributions within the muscle fiber. Also up to about 48 hours after in vivo transfection, no fluorescence was observed in the extracellular space of the muscle fibers. Further, no visible functional damage based on the electrical, mechanical, and Ca²⁺ signaling properties of the muscle fibers expressing EGFP or ECFP were observed post transfection.

Data collected 5 days after in vivo transfection of the plasmids encoding the fluorescent proteins showed similar results. FIG. 1 (panels A1 and A2) show optical assessments of the transfection efficiency of pEGFP-N2 in skeletal muscle. Panel A1 is an image illuminated by white light of an FDB muscle dissected 5 days after in vivo transfection with pEGFP-N2; whereas, panel A2 is an image from the same muscle when illuminated with monochromatic blue light (480 nm) and the fluorescence filtered with a 550 nm long pass filter. Both images in panels A1 and A2 were obtained with a 4 Mega pixels digital camera attached to a dissecting microscope. This demonstrated that EGFP expression occurred throughout the whole muscle.

FIG. 1, panel B2, was processed like panel B1, except the FDB muscle was from 5 days post transfection, instead of 12 hours post transfection. The microscope objective was an Olympus 10×, NA 0.25; the calibration bars represent 200 μm; and the 256 intensity levels of green span a fluorescence scale of 0-65,536 AU. For both B1 and B2. Panel C is a single high magnification TPLSM section image through a bundle of muscle fibers from the same muscle as that shown in panel B2. The tissue sample used in panel C was slightly stretched. The rectangular insert in panel C represents a section of the muscle fiber sample whereby addition data relating to the protein profile was measured. The graph in panel C showed peaks and breaks in the ordinate axis indicating levels of recombinant protein expression. The microscope objective was an Olympus 20×, NA 0.95 (Olympus XLUMPLANFL) and the length of the rectangle is 10 μm.

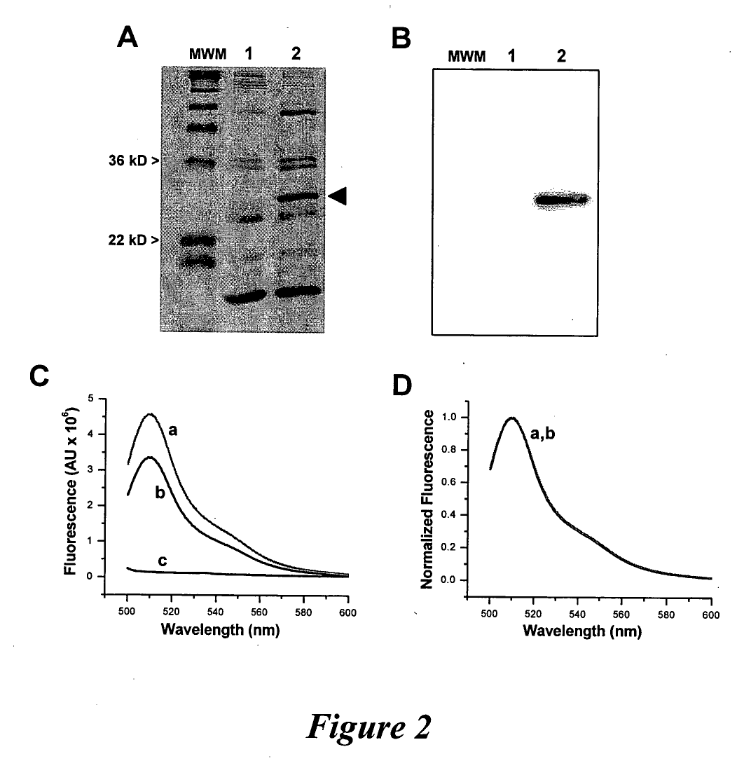

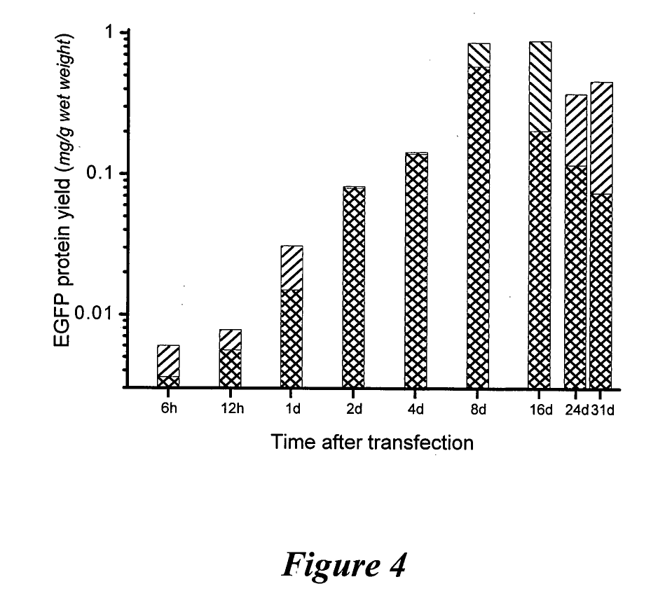

To determine the biochemical presence of the fluorescent proteins expressed in the FDB muscles, Western blot analysis of different muscle fiber homogenates was performed. FIG. 2 (panels A-D) shows that a protein with the approximate weight of GFP was observed. Panel A is a SDS-PAGE of supernatant fractions obtained from pEGFP transfected skeletal and no pDNA transfected muscle fibers. Muscle fibers which were transfected but with no pEGFP (mock control, lane 1) did not express an apparent protein about 26-27 kDa in size (arrowhead), as compared to the muscle fibers which had been transfected with pEGFP (lane 2). Each lane on the SDS-PAGE was loaded with about 7.3 μg of total protein, which represents anywhere from about 0.01% to about 2.2% of total recombinant protein, depending on the expression time period (see Table 1). The SDS-PAGE from panel A was transferred onto a nitrocellulose membrane and the Western Blot was probed using an anti-GFP antibody (Clontech). The Western blot analysis showed that the recombinant EGFP is expressed in large amounts even by about 2 to 4 days post transfection (see also FIG. 4).

To determine whether the expressed recombinant fluorescent protein contained fluorescent activity, fluorescence emission data was also collected from the transfected muscle fibers and compared with the mock control. FIG. 1 (panel C) shows the fluorescence emission spectra of 1:20 dilution of the supernatant obtained from a pEGFP transfected FDB muscle (trace a), an untransfected control muscle (or a muscle transfected with no pDNA; trace c), and 10 μg/ml commercial EGFP (trace b). Panel C shows GFP is expressed in those muscle fibers which were transfected with the plasmid encoding the GFP as compared to the untransfected control. The wet weight of the transfected muscle was 11.9 mg, and the total supernatant volume was 75.8 μl. Panel D, shows traces a and b normalized to their respective peaks at 508 nm and shown superimposed.

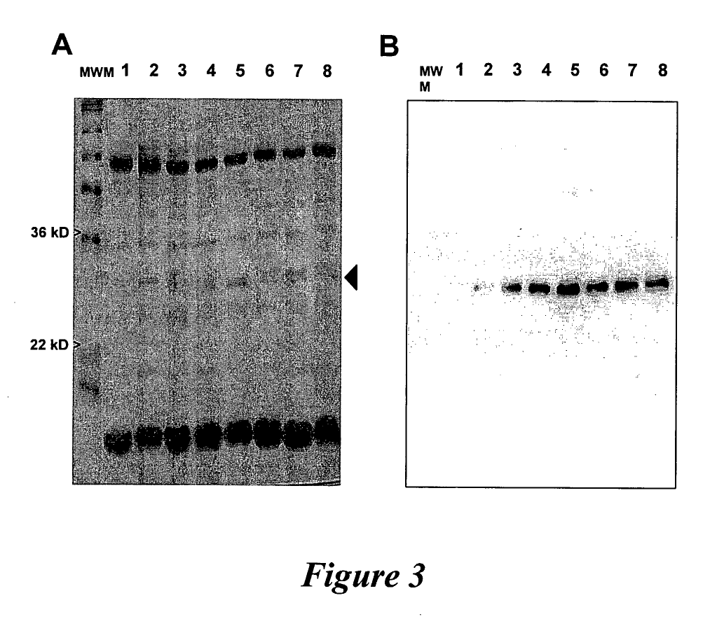

To determine the time course of recombinant EGFP expression in the transfected muscle cells, samples were collected at various time points post transfection and analyzed. FIG. 3 (panels A and B) shows the time course of expression of GFP in the transfected muscle cells. Panel A shows an SDS-PAGE of supernatant fractions obtained from lower limb muscles transfected with pEGFP. Muscle tissue was processed and samples were taken at 0.5, 1, 2, 4, 8, 16, 24 and 31 days post transfection. About 10 μg total proteins were loaded into each of lanes 1-8. The arrowhead indicates the position of a protein band corresponding to an apparent molecular weight of about 26-27 kDa. While the stained SDS-PAGE shows that the expression of the EGFP protein is comparable to other cellular proteins, transfer of the SDS-PAGE onto nitrocellulose and probing the Western Blot with an anti-GFP antibody showed that EGFP expression peaks about 8 days post-transfection. The Western blot analysis also showed that the EGFP does not break down into smaller polypeptide fragments, as smaller, lower molecular weight proteins were not detected by the anti-GFP antibody.

FIG. 4 is a bar graph of GFP protein yield, expressed in mg of EGFP per gram wet weight of lower limb muscle tissue, plotted as a function of the time after muscle transfection. Both axes are displayed in logarithmic scales. Experimental data was obtained in duplicates for each time point and drawn superimposed in the graph (cross marks show overlapping levels of protein in one set of data). Similar to the results shown in FIG. 3, GFP expression is observable 6 hours after transfection and peaks at about 8 days (about 0.8 mg/g) and continues though 16 days post transfection. Even at 31 days after transfection, GFP expression continues to be significant from about 0.6 mg/g to 1.6 mg/g).

The time course of GFP expression days after transfection is also shown in Table 2 below.

TABLE 2
Levels of protein extracted from supernatant fractions of
lower limb muscles at different periods after transfection

| | Transgenic | Total | | |
|---|---|---|---|---|
| Days | protein | transgenic | Total | Transgenic/ |
| After | concentration | protein | protein | total |
| transfection | (μg/ml) | (μg) | (mg) | protein (%) |
| 0.25 | 0.6 | 0.8 | 8.3 | 0.01 |
| | 0.8 | 1.2 | 8.6 | 0.01 |
| 0.5 | 0.9 | 1.3 | 7.6 | 0.02 |
| | 0.8 | 1.2 | 8.4 | 0.01 |
| 1 | 2.7 | 4.1 | 7.9 | 0.05 |
| | 5.7 | 8.5 | 8.4 | 0.1 |
| 2 | 13.3 | 35.9 | 16.0 | 0.2 |
| | 14.0 | 33.7 | 15.0 | 0.2 |
| 4 | 28.3 | 87.8 | 21.8 | 0.4 |
| | 30.7 | 76.7 | 21.2 | 0.4 |
| 8 | 141.5 | 339.7 | 15.4 | 2.2 |
| | 104.9 | 262.3 | 22.8 | 1.2 |
| 16 | 142.4 | 270.5 | 12.9 | 2.1 |
| | 57.0 | 256.5 | 24.7 | 1 |
| 24 | 38.1 | 87.7 | 12.1 | 0.7 |
| | 147.0 | 176.4 | 6.6 | 2.7 |
| 31 | 15.9 | 31.8 | 11.4 | 0.3 |
| | 103.6 | 217.6 | 12.5 | 1.7 |

### Discussion

The methods described show that mammalian skeletal muscle is a excellent system for transient heterologous protein expression. Although previous works from others demonstrate that muscle can be transfected by electroporation with DNA plasmids for the purpose of gene therapy, these studies fail to show that the level of expression in skeletal muscle can be a source for the large-scale production, extraction and purification of desired recombinant proteins.

There are many advantages to the methods described herein which are unobserved in other protein expression methods in the field. The combination of the various techniques provides for high protein yield. For example, the animals are anesthetized with gas anesthesia instead of injected agents. The procedures are designed to minimize both the dose of anesthesia and the periods when the animals are asleep, resulting in the animal's fast recovery from the transfection procedure. Typically, the animals are ambulatory for about 2-6 minutes, or for about 3-4 minutes, after the procedure, ensuring the normal use of transfected muscles. Also, injections of saline solutions (sterile) containing enzymes (pre-treatment) and plasmids into the muscles are done with fine, sterile needles, in order to avoid muscle damage and infections.

Further, for each electroporation protocol, 2 fine, sterile, acupuncture gold-plated needles are used to deliver current pulses to the muscles. This configuration was based on various studies using different configurations. For example, careful investigation of the geometrical features of each transfected muscle and orienting the needles in order to optimize the flow of current through the muscle increases electroporation efficiency. In addition, the amplitude, duration and repeatability of pulse delivery, using the efficiency of protein expression as the control is also optimized. The electroporation methods described herein have been designed to attain a balance between maximal pDNA transfer into the muscle cells (by current delivery) and minimal damage in order to ascertain the physiological intactness of the muscle fibers.

Although electroporation procedures as described herein help to produce large quantities of the proteins, but as compared with other electroporation publications, the protein yields described herein are significantly improved (e.g., about 1-4 orders of a magnitude, about 2-3 orders of magnitude, about 2 orders of magnitude, about 3 orders of magnitude, about four orders of magnitude and the like). Thus, the various optimizations facilitate increased protein expression and/or production. The invention is based on careful quantitation which was not previously reported. The invention describes protein yield that are about 10-fold or more as compared to the other reports. That is, the majority of the reports are 2-3 orders of magnitudes below ours. For example, the invention describes protein yields that are about 0.6 to 1.7 mg per gram of tissue. However, optimization of the methods as described herein, which are encompassed by the invention, can yield protein in about 0.5 to 3 mg per gram of tissue (e.g., about 1 mg to 2 mg per gram of tissue, about 0.6 to 1.6 mg per gram of tissue, about 1 mg per gram of tissue, about 2 mg per gram of tissue and the like).

Efficacy and efficiency of the in vivo transfection of skeletal muscle using the methods described herein was first evaluated by expressing heterologous EGFP. EGFP was chosen because EGFP location and concentration can be readily determined by microscopy methods, for example, TPLSM and transmission electron microscopy (TEM). Using TPLSM, the described methods herein show that GFP is consistently confined in the myoplasm. Although GFP is distributed throughout the fiber volume (FIG. 1), GFP is concentrated at the A band (FIG. 1 panel C), but to an extent not greater that 20% (FIG. 1 panel C insert). Based on the fact that the space among thick filaments is smaller than that among thin filaments, the GFP distribution suggests a preferential binding of GFP to A band proteins. Additionally, TPLSM studies showed that inclusion bodies known to occur in heterologous protein expression systems were never detected, regardless of the level of expression or the protein expressed. Baneyx, F. and M. Mujacic (2004) “Recombinant protein folding and misfolding in Escherichia coli,” Nat Biotechnol, 22(11):1399-1408.

All the molecular and biochemical studies confirm that GFP is expressed in skeletal muscle fibers after transfection. A stained SDS-PAGE showed a molecular weight protein corresponding to the molecular weight of GFP from the predicted amino acid sequence (i.e. 26-27 kDa; see FIG. 2, panel A). Further, the same apparent molecular weight protein was recognized by an anti-GFP antibody (Clontech; FIG. 2, panel B). To determine whether the GFP was properly folded, fluorescence emission spectra data was collected. The data confirmed that the recombinant EGFP in transfected muscle is indistinguishable from GFP expression obtained from bacteria (using an identical cistron; see FIG. 2 panel D). Hence, GFP was properly folded and has fluorescent activity.

The methods described herein show that, in contrast to the art, the expression of heterologous proteins expressed in mammalian skeletal muscle is in quantities (or units) amenable to quantitative comparison of the yields (i.e. mg/g). Dona, M., et al. (2003), “Functional in vivo gene transfer into the myofibers of adult skeletal muscle,” Biochem Biophys Res Commun, 312(4):1132-8; Wolff, J. A. et al. (1990), “Direct gene transfer into mouse muscle in vivo,” Science, 247(4949 Pt 1):1465-8; Mir, L. M. et al. (1999), “High-efficiency gene transfer into skeletal muscle mediated by electric pulses,” Proc Natl Acad Sci USA, 96(8):4262-7; and Hartikka, J. et al. (1996), “An improved plasmid DNA expression vector for direct injection into skeletal muscle,” Hum Gene Ther, 7(10):1205-17.

Also, in contrast to other studies, GFP expression and production in skeletal muscles as described herein is normalized with respect to the mass of muscle tissue (mg of protein/g wet weight). These units allow for easy comparison between the data described in this invention and that from other expression systems described by others. It was shown that 5 days after transfection, FDB muscles exhibited a large GFP expression yield of about 1.6 mg/g wet tissue, which is comparable to the about 1 mg/g of pellet generated by bacteria. Figueira, M. M. et al. (2000), “Production of green fluorescent protein by the methylotrophic bacterium methylobacterium extorquens,” FEMS Microbiol Lett, 193(2):195-200.

### EXAMPLE 2

### In Vivo Transfection of Proteins using Fluorescent Tags

This example demonstrated the use of fluorescent tags to gauge the level of efficiency of transfection of the proteins.

In order to investigate the pattern of the over-expression of other soluble proteins, adult mammalian skeletal muscle was transfected with DNA plasmids encoding for CFP-tagged (experiment), T7-tagged (control) recombinants of the muscle-specific soluble protein, β1a-subunit of the dihydropyridine receptor (β1a-DHPR), along side as GFP (control) alone. All plasmids were delivered by in vivo electroporation substantially as described above (e.g. delivering plasmid DNA in less than about 20 μg in mice to greater than about 150 μg in rats or larger animals; and from about 90V in mice to greater than about 200V in rats or larger animals).

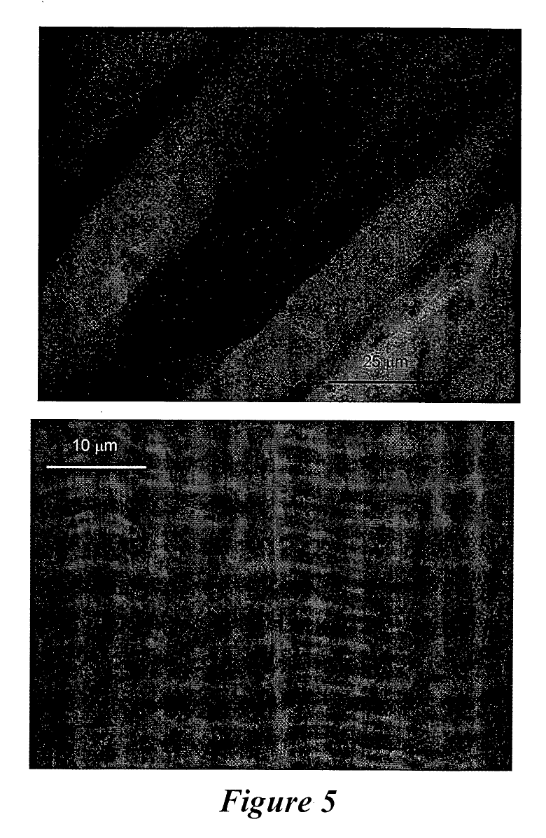

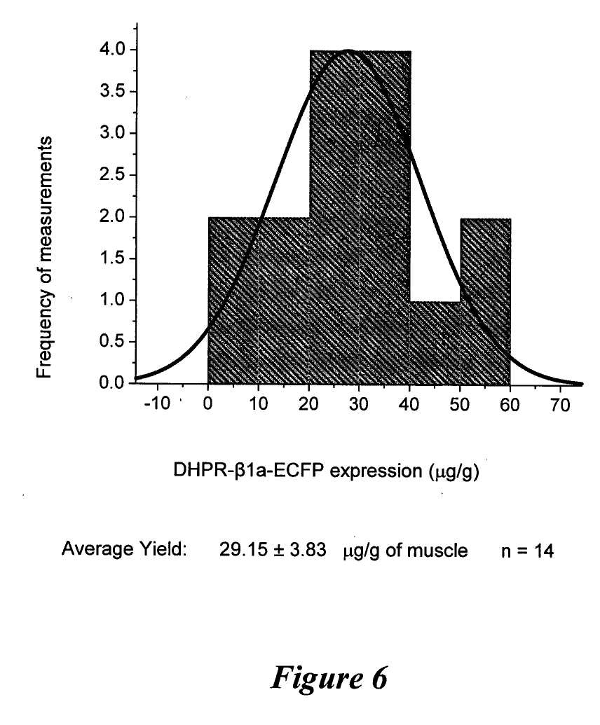

Using TPLSM, the expression and localization of fluorescently tagged CFP-β1a-DHPR was determined. About 12 hours post transfection, expression of recombinant CFP-β1a-DHPR displayed heterogenous levels of fluorescence in the muscle fibers, with the greater amount of fluorescence around the nuclei of the fibers (data not shown). Also, the ECFP-β1a-DHPR protein was circumscribed to narrow transverse bands in every sarcomere of the muscle fibers (panels A). However, TPLSM images from muscle fibers 5 days post transfection showed a more diffuse signal of the CFP-β1a-DHPR, approximating the pattern observed in CFP or GFP alone (FIG.5). The time dependent nature of CFP-β1a-DHPR localization was likely due to β1a-DHPR initial or early (about 12 to 24 hours post transfection) binding affinity to its transmembrane counterpart, β1a-DHPR, and as the expression of β1a-DHPR increases, there was a saturation of β1a-DHPR: β1S-DHPR, and the β1a-DHPR signal becomes more diffuse over time (after 5 days post transfection; see FIG. 5). pECFP-β1a-DHPR was also transfected in calf muscle and the amount of recombinantly expressed ECFP-β1a-DHPR protein was quantified as shown in FIG. 6.

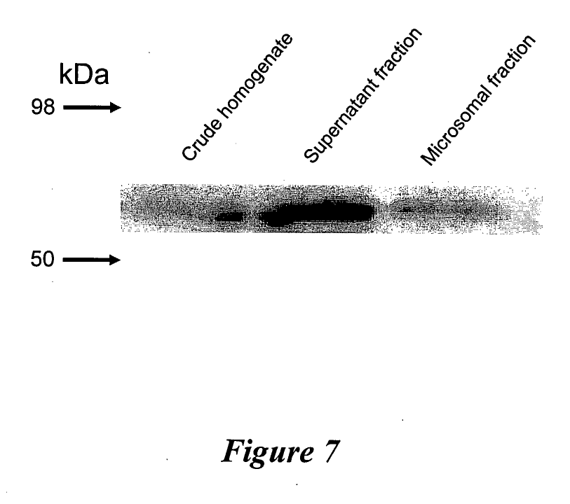

Further the T7-tagged recombinant β1a-DHPR protein (T7-β1a-DHPR), unlike the ECFP-β1a-DHPR or the EGFP or ECFP alone, was primarily found in the muscle fiber microsomal fractions, instead of the soluble fractions. This was confirmed by Western blot analysis using anti-T7 monoclonal antibody (Novagen) (FIG. 7).

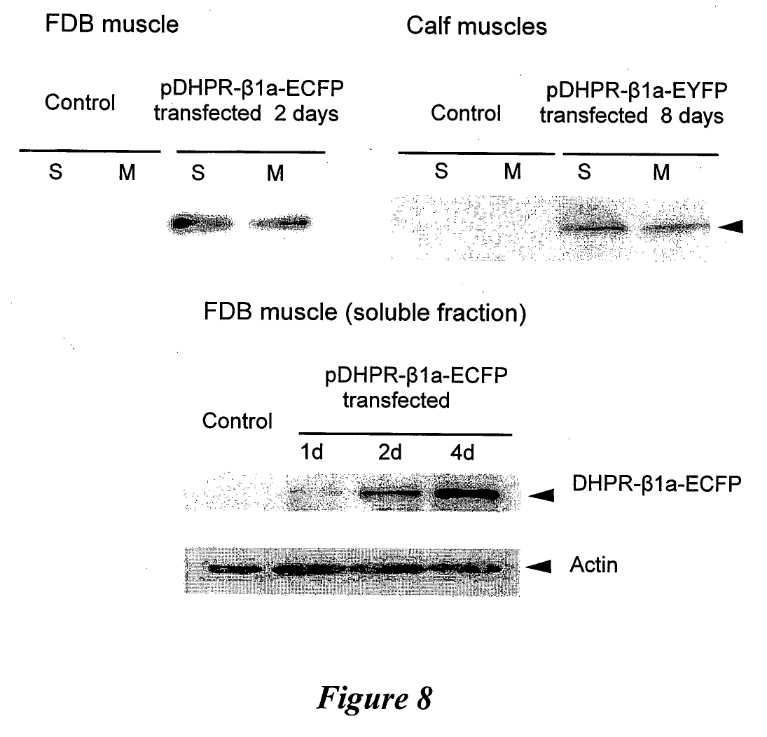

pEYFP-β1a-DHPR was also constructed and transfected in both calf muscles and FDB muscles. Expression of the recombinant EYFP-β1a-DHPR and ECFP-β1a-DHPR proteins was both detected using the anti-YFP and CFP antibodies as show in FIG. 8. This was compared to actin (lower panel), an endogenous muscle protein, which was constitutively expressed (or constant) at 1, 2 and 4 days post transfection. Thus, the recombinant proteins were expressed due the in vivo transfection methods and expression of the protein occurs in a time dependent manner.

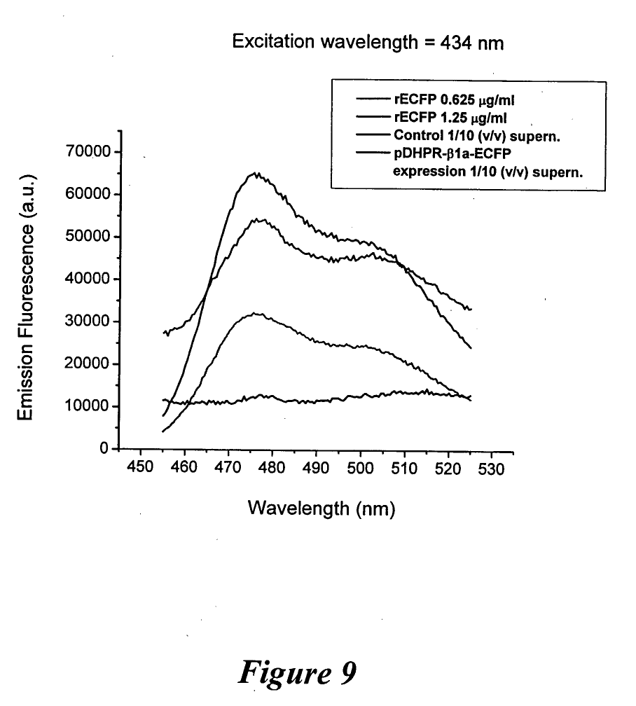

Emission spectra data was also collected from recombinant ECFP-β1a-DHPR protein transfected in calf muscles. Calf muscle fibers transfected with pEYFP-β1a-DHPR expressed fluorescent EYFP-β1a-DHPR protein had comparable emission spectra as compared to that of purified recombinant ECFP (rECFP; FIG. 9).

The methods described herein show that fluorescent tags (e.g. EGFP, ECFP and EYFP) can be attached to heterologous soluble proteins (e.g., β1a-DHPR). The fluorescent tags do not affect localization or disrupt proper protein folding of the recombinant protein. For example, experiments using soluble cytosolic proteins (GFP and CFP), muscle specific soluble protein (β1a-DHPR), and transmembrane proteins(GFP-α1S-DHPR, RyR1 and Shaker channel; see Example 3) showed that localization of the proteins were different in all three protein types and that localization of the recombinant proteins was similar to that observed for the native proteins.

### EXAMPLE 3

### In Vivo Transfection and Production of Large Quantities of Transmembrane and Cytosolic Proteins

This example demonstrated the in vivo transfection and production of transmembrane and cytosolic (soluble) proteins.

In vivo electroporation of compositions consisting of various DNA (or cDNA) plasmids encoding for α1S-DHPR, RyR1, and Shaker channel transmembrane proteins was performed substantially similar to that described above, using fast and slow twitch muscle fibers from young anesthetized mice (e.g. extensor digitorum longus (EDL), soleus, tibialis anterior (SA), and flexor digitorum longus (FDB) and flexor digitorum quinti (FDQ)). Two (2) types of solutions were injected subcutaneously into the legs or footpads of the animals: A solution of 2 mg/ml hyaluronidase dissolved in pharmaceutical grade sterile saline filtered (after mixing) with 0.2 μm pore sterile filters; and an experimental solution containing 2-5 μg/μl of cDNA plasmids (e.g., α1S-DHPR) encoding soluble or transmembrane proteins, some which are listed below in Table 3.

TABLE 3
Plasmid vectors for use in in vivo transfection

| Protein | Vector | Manufacturer | Promoter | Tag position (bp) | Species |
|---|---|---|---|---|---|
| EGFP | pEGFP-N2 | Clontech | CMV | | Aequorea Victoria |
| ECFP | pECFP-N1 | Clontech | CMV | | Aequorea Victoria |
| EGFP-Shaker | pCDNA3 | Invitrogen | CMV | 1042-1758 | Drosophila melanogaster |
| YFP-RyR1 | pCI-neo | Promega | CMV | N-term | Mouse skeletal muscle |
| ECFP-RyR1 | pCI-neo | Promega | CMV | N-term | Mouse skeletal muscle |
| GFP-α1s-DHPR | | Proprietary | CMV | N-term | Rabbit skeletal muscle |
| ECFP-β1a-DHPR | pECFP-C1 | Clontech | CMV | N-term | Mouse skeletal muscle |
| β1a-DHPR-ECFP | pECFP-C1 | Clontech | CMV | C-term | Mouse skeletal muscle |
| β1a-DHPR-EYFP | pEYFP-N1 | Clontech | CMV | C-term | Mouse skeletal muscle |
| T7β1a | PSG5.1 | Stratagene | T7/SV4 | N-term | Mouse skeletal muscle |

The experimental procedure was divided in two (2) phases. During the first phase, in order to gain access to the region where the specific muscle was located, about 10-30 μl of the hyaluronidase solution was injected subcutaneously using a 30-33 gauge sterile needle. Care was taken to avoid penetration of the muscle with the needle and the solution was injected slowly in order to avoid muscle damage and to ensure that the solution ran freely in the interstitial fluid. After recovery from anesthesia, mice were allowed to freely move in the cage for a period of 1-2 hours. Following this period, the animal was again anesthetized and injected subcutaneously, at the same site of the hyaluronidase injection, with about 10-30 μl of the plasmid containing cDNA sterile solution, taking the same precautions as in the first injection. Ten minutes after the injection, two sharp platinum needles (30 gauge) were placed through the skin on each end of the muscles of interest and about 10-30 stimulus pulses (50-100 V, field strength 50-100 V/cm, 20 ms duration/each) were applied and delivered at a rate of 1 Hz. A surgical dissecting microscope was used to verify the proper placement of the electrodes. The stimulus pulses were generated by a Grass S88 medical stimulator and monitored in an oscilloscope. At the end of the stimulation period, the animal was allowed to recover completely and transferred to the cage with periodic assessment of health.

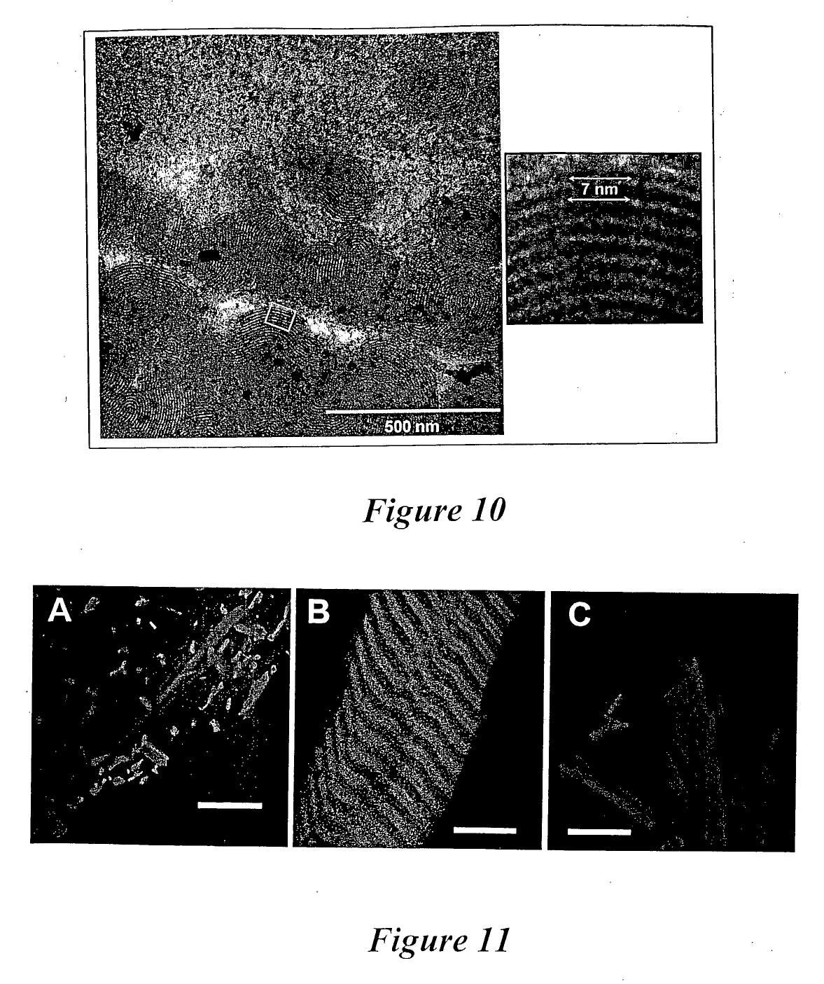

Using TPLSM and TEM, expression of the fluorescently-tagged functional recombinant α1S-DHPR, the mammalian ryanodine receptor (RyR1), and the Shaker K channel proteins from Drosophila were analyzed. (pYFP-RyR1 and pGFP-α1S-DHPR were gifts from P. Allen; and pEGFP-Shaker, a gift from F. Bezanilla and R. Blunk). Transmission electron microscopic analysis showed that in all three cases the majority of the fluorescence bodies seen in TPLSM were localized in the extracellular (or interstitial) space surrounding the muscle fibers (FIG. 10).

Although some of the post transfected muscle fibers have variable levels of intracellular expression of GFP-α1S-DHPR protein, the great majority of the recombinant GFP-α1S-DHPR protein is found in irregularly shaped bodies localized in the extracellular space between the muscle fibers (FIG. 11). These extracellular fluorescent bodies are highly dense aggregates of the GFP tagged membrane protein in a quasi-crystalline assortment associating with endogenous muscle lipids. The recombinant GFP-α1S-DHPR protein aggregates have been processed exported by the muscle fibers during the course of the 5 days post transfection; while the fibers themselves retain a relatively small proportion of the newly synthesized protein. In addition, the presence of properly folded protein in the extracellular bodies is implied by the presence of fluorescence itself, since this property is by necessity associated with correctly folded GFP.

TPLSM images demonstrated that although there is a great variability in the size of each of the fluorescent bodies, they were, in general, quite large (FIG. 11). Hence, their large size rejects the possibility that they may represent phagocytes containing the fluorescent protein inside (see panel A). The TPLSM images also show that the fluorescent bodies have very irregular shapes displaying at times sharp angles which suggest that they conform to structural determinants of densely packed proteins (FIG. 11 panel C).

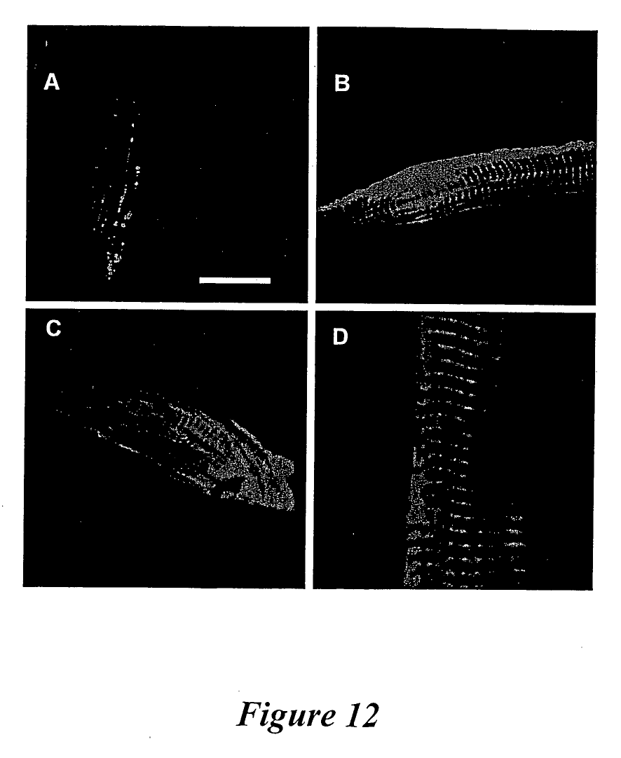

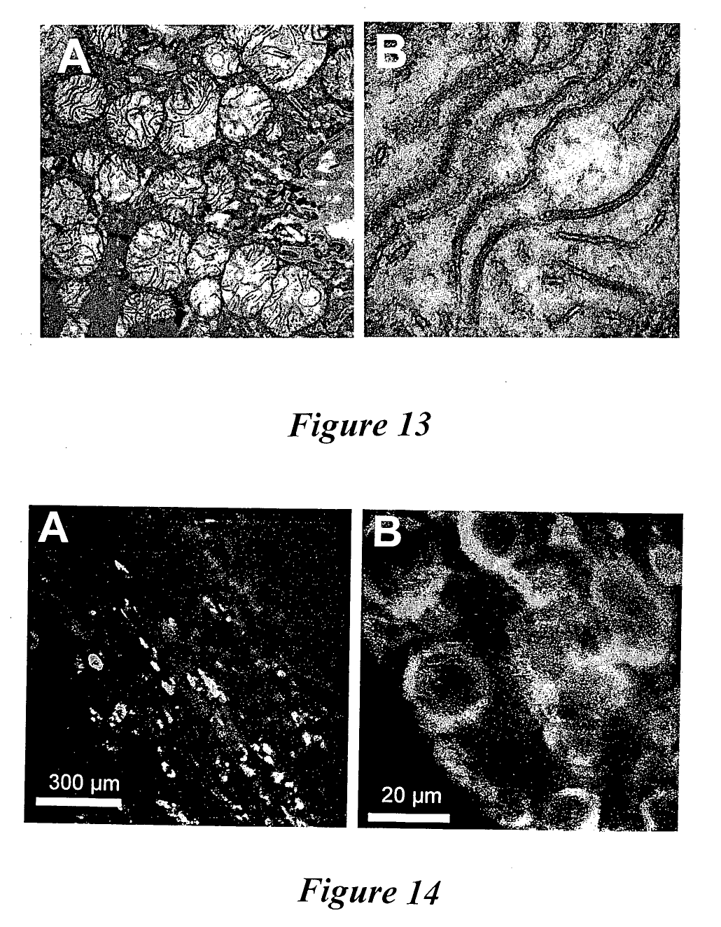

TPLSM images of the expression of recombinant EGFP-Shaker channel proteins also showed that similar to recombinant GFP-α1S-DHPR proteins, DGFP-Shaker proteins were localized in extracellular fluorescent bodies and in the membranes of the muscle fibers (FIG. 12); although the fluorescence in the T-tubules and internal muscle fiber membranes is more conspicuous than that observed for GFP-α1S-DHPR proteins. Again, the absence of the fluorescent proteins in the myoplasm, and its specific localization in the membrane systems suggests that the expressed recombinant protein was correctly folded and targeted. Transmission electron microscopy inspection of the subcellular region of the transfected muscle fibers revealed alterations of the internal membranes of the mitochondria (FIG. 13).

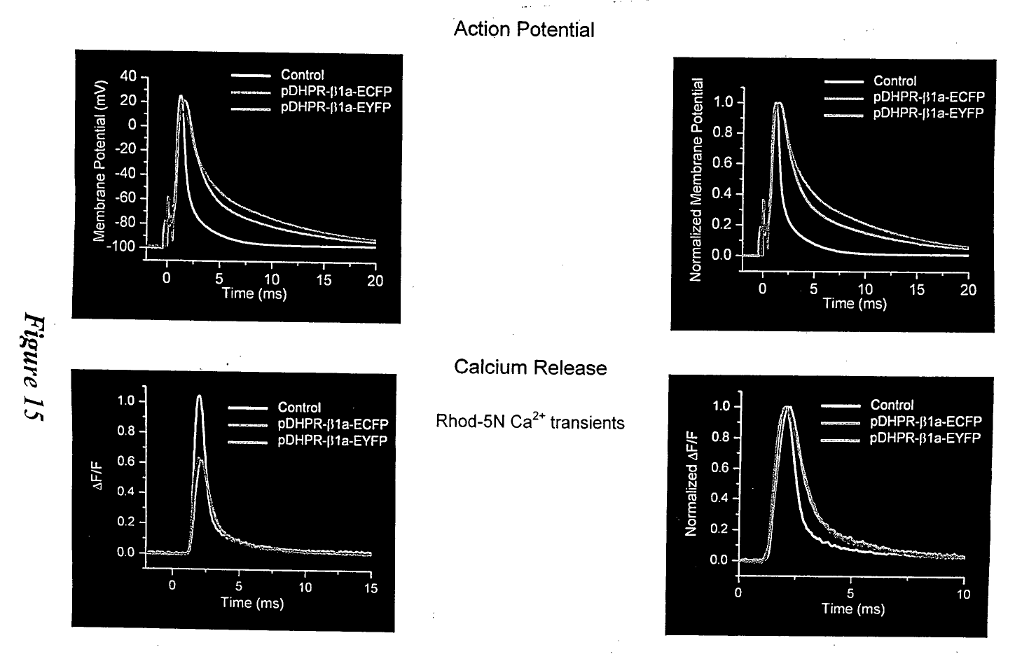

The expression of YFP-RyR1 channels resulted in fluorescent aggregates in the extracellular space around the muscle fibers (FIG. 14). Levels of protein expression for YFP-RyR1 were less than that observed for either of the other two transmembrane proteins (i.e. GFP-α1S-DHPR and EGFP-Shaker). This is part could be due to the size of the encoding polynucleotide sequence, which is a larger cistron than the other two transmembrane proteins. Unlike, GFP-(α1S-DHPR and EGFP-Shaker localization, YFP-RyR1 was localized in globular structures, which did not resemble the quasi-crystalline appearance of GFP-α1S-DHPR, or the solid appearance of EGFP-Shaker (FIG. 14).

Depending on the protein, the muscle fibers showed variable levels of expression in intracellular organelles such as T-tubules and mitochondria. Moreover, visual inspection revealed no apparent damage in most of the fibers of transfected muscles. It is submitted that expressed fluorescent proteins are protein/lipid aggregates exported by the muscle fibers during the post-transfection period (1-5 days), while the fibers themselves retained a variable proportion of newly synthesized protein. Furthermore, the intense fluorescence of the extracellular protein/lipid aggregates suggests that the fluorescent tag proteins were properly folded. Hence, this suggests that the recombinant transmembrane proteins are also properly folded; since it is unlikely that only the fluorescent tag has proper tertiary structure while the rest of the encoded protein is disorganized. Electron microscopy further showed that the extracellular fluorescent objects displayed a well-organized arrangement of the transmembrane proteins.

Other recombinant membrane proteins have been transfected into muscles cells using methods substantially as described herein, including: 1) cardiac Na/Ca2+ exchanger (NaX), e.g., N-tagged with EYFP (EYFP-NaX) and center tagged with EGFP (NaX-EGFP-NaX); 2) N-tagged EGFP sarcospan (a muscle sarcolemmal protein) (EGFP-sarcospan); 3) Farnesilated EGFP (F-EGFP; Clontech); and 4) N-tagged EGFP-α1S-DHPR (subdloned into a Clontech plasmid in our laboratory). Further, FRET measurements using the methods substantially as described herein were also possible using muscle cell transfected with F-EGFP. While the N-tagged EGFP-α1S-DHPR plasmid replaces the pGFP-α1S-DHPR kindly donated by Grabner and Beam because the EGFP tag is brighter than the GFP tag.

Similarly, other recombinant soluble proteins have been transfected into muscles cells using methods substantially as described herein, including N-tagged calpain (the muscle isoform called C3) (EGFP-C3)

Thus, the methods described herein are useful to express large quantities of not only heterologous cytosolic proteins, but heterologous transmembrane proteins as well. Expression of the fluorescently tagged α1s-DHPR, RyR1 and Shaker channel transmembrane proteins demonstrate the efficacy of the in vivo transfection methods, and the use of muscle as mammalian host cell model.

### EXAMPLE 4

### Transfected Skeletal Muscle Cells Maintain Normal Function

This example demonstrated that recombinant protein expressed in transfected skeletal muscle cells retain their physiological function, specifically, the function to maintain an action potential.

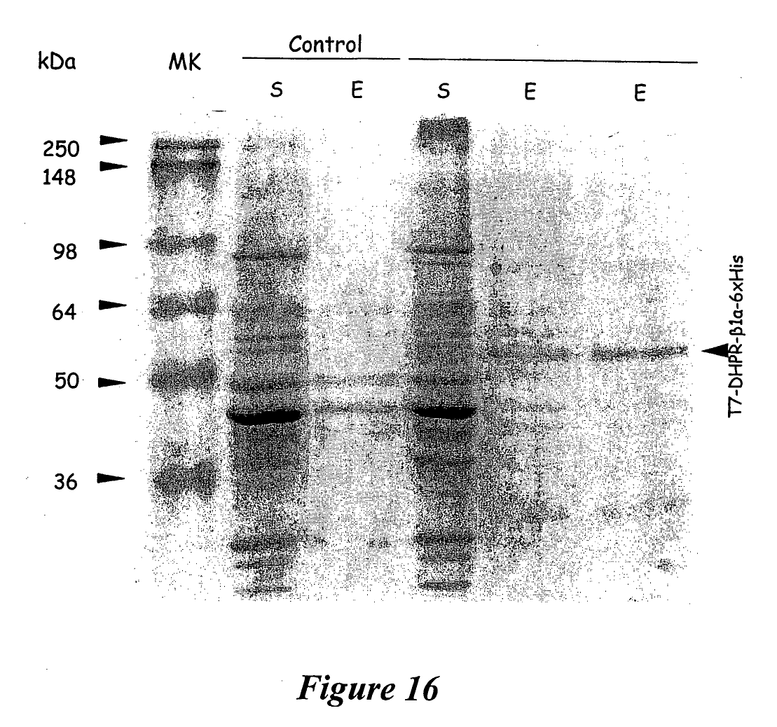

To determine whether skeletal muscles transfected with the various transmembrane and/or soluble heterologous proteins affected the physiological function of the muscle to produce action potentials, electrophysiological data was recorded. A recording from a single point showed that the various stages of an action potential (depolarization and repolarization) were intact for skeletal muscle cells transfected with pEYFP-β1a-DHPR and pECFP-β1a-DHPR, as well as the control. That is, all three recordings showed similar typical schematic of an action potential (FIG. 16).

To determine whether the role of Ca²⁺ in transfected skeletal muscle was disrupted, Ca²⁺ release was detected using Rhod-5N Ca²⁺ transients. Ca²⁺ recordings were collected for pEYFP-β1a-DHPR and pECFP-β1a-DHPR transfected muscles. Muscle contraction and function as measured by monitoring and recording of Ca²⁺ levels was normal for the transfected muscle fiber cells as compared to the untransfected or control muscle fiber cells (FIG. 15 and Table 4).

TABLE 4
Expression of recombinant β1a-DHPR: Effects
on the Action Potential and Calcium Release

| | Action Potential | | Calcium Release | |
|---|---|---|---|---|
| | Amplitude | FDHM | | FDHM |
| | (mV) | (ms) | (ΔF/F)ₚₑₐₖ | (ms) |
| Control | 122.83 ± 1.71 | 1.11 ± 0.05 | 0.90 ± 0.04 | 1.10 ± 0.03 |
| pECFP-β1a-DHPR | 111.47 ± 0.85 | 1.94 ± 0.09 ** | 0.70 ± 0.03 * | 1.52 ± 0.05 ** |
| pECFP-β1a-DHPR | 121.72 ± 3.22 | 2.36 ± 0.26 ** | 0.67 ± 0.04 * | 1.52 ± 0.09 * |

* Statistical significance (P < 0.05);
** Statistical significance (P < 0.0001); Student T-test

Thus, the above show that the in vivo transfection methods described herein of recombination proteins does not affect the electrophysiological function of the muscle cell as evidenced by the fact that transfected muscle fiber cells showed similar electrophysiological action potential recordings and Ca²⁺ release and sequestration during muscle contraction. Therefore, the methods described herein do not affect the function and integrity of the muscle fiber.

### EXAMPLE 5

### Protein Purification of Recombinantly Expressed β1a-DHPR Protein in Skeletal Muscle

This example demonstrated that recombinantly expressed proteins in in vivo transfected skeletal muscle cells are of sufficiently large quantity allowing for protein purification.

Crystallization requires on the orders of about 10 to 20 mg/ml of pure protein, while at the same time keeping the total volume of the pure protein solution to a small volume (e.g. less than about 400 μl). Thus, for crystallization purposes, about 2-4 mg or pure protein is sufficient.

A plasmid containing the nucleic acid encoding β1a-DHPR was operably linked to the T7 promoter at the 5′ N-terminus, and a 6 Histidine tag at the 3′ COOH-terminus. In vivo transfection of the protein in muscle fibers was performed substantially as described above and muscle cell homogenates made. The recombinantly expressed protein was purified using a TALON Co2+ column per the manufacturer's recommendations (Clontech). FIG. 16 was stained SDS-PAGE containing various fractions (S and E, supernatant and expressed, respectively) from untransfected (control) and transfected muscle fibers. A protein of about 53-54 kDa was observed after purification and this corresponds to the approximate molecular weight of β1a-DHPR plus T7 and a 6 Histidine tag.

Thus, the methods described herein are capable of producing large quantities of a recombinant protein for protein purification.

All together, the methods described and the data presented herein provide for a means of producing large quantities of expressed recombinant transmembrane and cytosolic proteins. The methods described herein are improved over other methods by orders of magnitude. For example, Umeda et al. (2004) describe production of about 20 ng/g wet weight of muscle reported. Umeda, Y. et al. (2004), “Skeletal muscle targeting in vivo electroporation-mediated HGF gene therapy of bleomycin-induced pulmonary fibrosis in mice,” Lab Invest, 84(7):836-44). In contrast, the methods and data above show that up to 0.8 mg/g of protein (FIG. 4) is possible. The present invention describes methods for producing recombinant proteins which can make up about 2.7% of the total cellular protein (Table 1). This amount is sufficient for various biological studies including protein purification and use of the pure protein for crystallization analysis.

Moreover, the methods described herein can be performed in either large or small muscle mass. The invention describes transfected FDB, lower limb, as well as upper limb (quadricep and hamstring) muscles. Hence, one skilled in the art will understand that the various methods and materials described herein can be scaled and tailored for use with a particular muscle size. For example, the total amount of GFP collectable per animal reached significant levels, for example, on the order of about 0.8 mg (FIG. 4). These results are encouraging since even larger muscle groups (e.g. quadriceps and hamstrings) can be transfected using substantially similar in vivo transfection methods as described herein. Furthermore, methods described herein provide a model for transfecting equivalent muscles in larger animals, e.g., preliminary tests performed using rat skeletal muscle are promising and will increase the expected yield of total protein by ten-fold.

Another advantage of the methods described herein is that they are easy to implement and relatively inexpensive, thus making them more cost effective. Since the protein expression occurs in live animals, the requirements for tissue culturing and the incubating machinery that other mammalian expression systems utilize, are not necessary. In addition, the use of naked DNA plasmids, instead of viral vectors, allows expressing large transcripts in a biologically safe fashion.

Thus, the above advantages make muscle an ideal expression system for applications in the biotechnological industry where it is imperative to express functional proteins rapidly and efficiently with the proper post-translational modifications, including protein folding and glycosylation. The mammalian skeletal muscle system intrinsically complies with these requirements since it is based on the use of mammalian cells.

The invention describes the in vivo transfection and recombinant expression of various transmembrane and cytosolic heterologous proteins. However, other proteins are within the scope of the invention, and one skilled in the art would understand that these teachings can be used to express other recombinant proteins other than that described herein. These studies also confirm that post-translational modifications, trafficking, and targeting of the proteins to the right cellular compartment, are found in the methods described herein.

Although the invention has been described with reference to the above examples, it will be understood that modifications and variations are encompassed within the spirit and scope of the invention. Accordingly, the invention is limited only by the following claims.

1. An in vivo method for producing elevated levels of transmembrane protein in a mammalian cell as compared to standard methods, comprising:
contacting a nucleic acid sequence encoding the transmembrane protein operably linked to regulatory elements with a muscle cell of a subject;
introducing the nucleic acid sequence into the cell by electroporation, wherein expression of the transmembrane protein is by endogenous translation of the nucleic acid sequence, thereby producing more transmembrane proteins in a mammalian cell as compared to standard methods.

2. The method of claim 1, wherein the muscle cell is a skeletal muscle cell.

3. The method of claim 1, wherein the transmembrane protein is a channel, pump, or receptor transmembrane protein.

4. The method of claim 1, wherein the contacting comprises injection.

5. The method of claim 1, wherein the mammal is human.

6. The method of claim 1, wherein the protein is selected from the group consisting of α1S-DHPR, RyR1, Shaker K channel, the cardiac Na/Ca2+ exchanger (NaX) and sarcospan proteins.

7. An in vivo method for producing elevated levels of cytosolic protein in a mammalian cell as compared to standard methods, comprising:
contacting a nucleic acid sequence encoding the transmembrane protein operably linked to regulatory elements with a muscle cell of a subject;
introducing the nucleic acid sequence into the cell by electroporation, wherein expression of the transmembrane protein is by endogenous translation of the nucleic acid sequence, thereby producing more cytosolic proteins in a mammalian cell about 2-3 orders of magnitude more as compared to standard methods.

8. The method of claim 7, wherein the muscle cell is a skeletal muscle cell.

9. The method of claim 7, wherein the contacting comprises injection.

10. The method of claim 7, wherein the mammal is human.

11. The method of claim 7, wherein the protein is selected from the group consisting of β1a-DHPR and calpain proteins,.

12. An in vivo method for expressing elevated protein levels from endogenous expression of a polynucleotide encoding the protein in a skeletal muscle cell as compared to standard methods, comprising:
a) contacting the skeletal muscle cell with a tissue permeability enhancing agent;
b) contacting the skeletal muscle cell with a recombinant expression vector encoding the transmembrane protein operably linked to a suitable promoter; and
c) applying an electrical stimulus to the muscle cell.

13. The method of claim 12, wherein the protein is a transmembrane protein.

14. The method of claim 13, wherein the transmembrane protein is a channel, pump, or receptor transmembrane protein.

15. The method of claim 14, wherein the protein is selected from the group consisting of, α1S-DHPR, RyR1, Shaker K channel, the cardiac Na/Ca2+ exchanger (NaX) and sarcospan proteins.

16. The method of claim 12, wherein the protein is a soluble protein.

17. The method of claim 16, wherein the soluble protein is selected from a group consisting of β1a-DHPR and calpain proteins,.

18. The method of claim 12, wherein the contacting comprises direct injection.

19. The method of claim 12, wherein the tissue permeability enhancing agent is hyaluronidase, chondroitinsulfatase, or a combination thereof.

20. The method of claim 12, wherein the transmembrane protein is a channel, pump, or receptor transmembrane protein.

21. The method of claim 1, wherein the expression vector further comprises a fluorescent and Histidine tag.

22. The method of claim 21, wherein the fluorescent tag is green fluorescent protein or cyan fluorescent protein.

23. The method of claim 22, wherein the Histidine tag is a 6 His tag.

24. The method of claim 12, wherein the contacting comprises electroporation.

25. The method of claim 12, wherein the tissue permeability enhancing agent is hyaluronidase, chondroitinsulfatase, or a combination thereof.

26. The method of claim 12, wherein the quantity of protein expressed is on the orders of mg per gram of wet weight.

27. A transfected mammalian muscle cell capable of producing elevated levels of protein as compared with standard transfected muscle cells, comprising, a muscle cell transfected with a nucleic acid encoding the protein.

28. The muscle cell of claim 27, wherein the protein produced is about 0.5 mg to 2 mg per gram of tissue.

29. The method of claim 1, wherein the level of protein is greater than two times the level of protein in standard methods.

30. The method of claim 7, wherein the expression vector further comprises a fluorescent and Histidine tag.

31. The method of claim 12, wherein the expression vector further comprises a fluorescent and Histidine tag.

32. The method of claim 30, wherein the fluorescent tag is green fluorescent protein or cyan fluorescent protein.

33. The method of claim 31, wherein the fluorescent tag is green fluorescent protein or cyan fluorescent protein.

34. The method of claim 32, wherein the Histidine tag is a 6 His tag.

35. The method of claim 33, wherein the Histidine tag is a 6 His tag.
